(19) United States
(12) Patent Application Publication
(10) Pub. No.: US 2016/0166195 A1
(43) Pub. Date: Jun. 16, 2016

(54) Energy and Food Consumption Tracking for Weight and Blood Glucose Control

(71) Applicants: Katarzyna Radecka, Verdun (CA); Zeljko Zilic, Verdun (CA)

(72) Inventors: Katarzyna Radecka, Verdun (CA); Zeljko Zilic, Verdun (CA)

(21) Appl. No.: 14/570,070

(22) Filed: Dec. 15, 2014

(51) Int. Cl. A61B 5/00; G09B 19/00; A61B 5/11

(52) U.S. Cl. CPC A61B 5/486; A61B 5/112; A61B 5/4866; A61B 5/743; G09B 19/0092

(57) ABSTRACT

The present invention relates to the area of lifestyle devices and applications, particularly to daily trackers of activities, such as pedometers used for exercise monitoring, and working in collaboration with tools analyzing and planning the food intake and amount of exercises to keep or obtain the desired weight. This invention aims at better controlling: fitness level, food consumption, blood glucose level and weight of a person throughout the day. For daily and weekly tracking of the weight, the ingested and expended calories are reported. Calories meal goals are adjusting daily reflecting the over the goal exercise levels as well as mismatch in food consumed and meal goals. The method for better blood glucose control predicts the effects of the food ingested on raising a post-meal blood glucose level, and suggests a suitable timing and the duration of the exercise following the food intake to minimize a post-meal blood glucose peak.

### FIELD OF THE INVENTION

This invention relates to the area of integrated lifestyle devices used for continuous monitoring of exercise, food intake, weight-loss tracking and improvements in blood glucose management. In particular, it proposes a personalized method for managing weight and fitness level. Further, this invention explores the concept of minimization of post-prandial glucose spike through appropriately timed exercises. As a result, through personalized weight-loss management, exercise and food tracking, as well as post-meal blood glucose control, the proposed solution contributes to the reduction of the risk of developing high blood sugar-related illnesses, and better managing the existing adverse health conditions related to obesity or high blood sugar.

### BACKGROUND OF THE INVENTION

Maintaining active lifestyle and suitable diet is critical to health and wellness regardless of age. Typical daily physical activities comprise habitual actions and different levels of organized exercise routines. The recommended level of exercises depends on age and gender [1], [2]. However, a truly compete picture of daily lifestyle activities emerges only when the energy profile of physical activities and consumed food is continuously gathered and aligned in time. Portable activity and food intake tracking can be effectively realized by smartphones and wearable electronic devices such as smart watches, bands and alike.

Accounting for food intake with electronic devices is more time-intensive for individuals than tracking the exercise. Further, relating the effects of the food and exercise to the fitness level and the impact to the weight requires both food and daily activities to be examined accurately. Therefore, devices and methods that enable users to effectively and accurately record food and activity are much more useful than the activity trackers alone.

The common ways to measure the intensity and duration of daily activities are by means of accelerometer-based pedometers [3], which should be equally accurate for all users regardless of their age, gender and weight. Together with personal characteristics (weight, height, age, gender), the duration and the speed of the movement contribute to expending the energy [4], [5]. Therefore, pedometers should account accurately for: step count, type of movement, speed, distance, and energy consumed. It has been noted that the pedometer accuracy depends on personal characteristics (weight, height, age, gender) as well as the speed of motion, and, for instance, overweight persons and children often report inaccurate pedometer readings [1], [2].

Weight-loss impact accounting consists of tracking both the food intake and the exercise level, and contrasting that information to the weight that is monitored either automatically, by means of a networked scale, or manually, by a user.

Various methods have been proposed to account for the metabolic impact of the food and exercise, and to take a correcting action to reach weight and exercise goals. In [6], a system is presented that aids the weight loss based on the food and exercise data. Patent [7] provides the way to time and regulate the glucose release for the best muscle performance, which primarily applies to sports. The method from [8] provides a generic way to predict the effects of the food and exercise on the blood glucose level, and the corrective action, mainly in terms of insulin injection. The closed loop control system is presented in [9] that keeps blood glucose within acceptable bounds. The method from [10] deals with techniques to suppress appetite while maintaining the exercise performance. The method has been disclosed in [11] that combines food planning and the exercise targeted to reduce the blood glucose spikes. The resulting exercise is shown to be effective, but not too strenuous. Blood glucose spikes have further been shown to depend on the Glycemic Index (GI) and Glycemic Load (GL) of the meals. Larger GL and GI indices indicate higher contribution to post-prandial (i.e., following the meal) blood glucose (BG) peaks.

### BRIEF SUMMARY OF THE INVENTION

This invention presents methods for comprehensive monitoring of daily activities such as exercises and food consumption, and analyzing their impact on individual's weight and blood glucose management.

Tracking of physical activities such as walking and running is performed automatically using data collected by accelerometers and then processed by a set of pedometer algorithms [3]. To accurately assess the suitability of the activity level, the overall daily physical movements, as well as singular exercises, are tracked and evaluated against user-set goals.

Daily caloric intake is obtained from the food nutrient data. Daily food calories recommendation is determined based on the user metabolism, weight-loss daily caloric deficit and exercise caloric goal. The system tracks food entries of up to 6 daily meals. Users can further specify the caloric goal for each of the meals, given the total food caloric goal determined by the tool. Although meal calories goals are long-term goals, they are adjusted based on a particular day specifics. If the food consumed in previous meals deviates from the goals, or the exercise level exceeds the goal, the goal for a given meal can be adjusted. Such meal goal adjustments help stay as close as possible to the daily caloric balance through the day and hence maintain the healthy lifestyle and guarantee a success of a weight-loss program.

The carb intake goals are set for the whole day, as well as for the individual meals. Unlike total calories goal, which is automatically determined in [0009], the carbs goals are based on user's preferences or medical advice. For instance, to keep their blood sugar levels manageable diabetics avoid taking too many carbs in the morning, while taking sufficient calories for daily activities. Therefore, the user pre-sets the carbs goal for the day and for individual meals that are to be checked throughout the day.

Food data collection requires a direct user's involvement. For the accurate analysis of daily caloric expenditure, the user is asked to scrupulously enter each idem consumed in a given meal. To minimize the effort and to facilitate better food data analysis, the system provides extendible Food Library that comprises initially over 3600 popular food items. In addition to the standard nutrition facts (carbohydrates, fiber, fat, proteins, sodium, cholesterol), each food item is matched with its glycemic index and glycemic load for improved estimate of the food intake effects.

For all daily meals entered to the system, users are given the complete nutrients profile, including the composite glycemic load as well as the graph illustrating the percentage distribution of meal calories among fats, carbs and proteins. Further, users are informed, whether the meal calories and carbs contents were on target with the assumed meal goals.

For each food item, as well as for a complete meal, the user can select two options of reporting the consumed carbs. First, the available carbs are obtained as a total amount of carbs in the meal (food item). In the second option, the amount of available carbs excludes fiber from the total amount of carbs. In addition to the available carbs, the total amount of carbs (with fiber included) is also reported. The option of reporting carbs without fiber is preferred by users on special diet and those who need to administer insulin.

The nutrition profile of a meal, including calories, proteins, and the glycemic index and load is used to estimate the amount of exercises needed for the user to expend (often referred to as “burn”) the food calories. Such Caloric Impact includes examples of walking and running routines, detailing the speeds, number of steps and duration of the exercise.

Further, the impact of the carbs on the post-meal blood glucose is predicted based on the nutrient profile. To remedy the chances of the post-meal blood glucose to reach unhealthy levels, the tool issues a recommendation with the time-after-meal and severity of the exercises.

Given the complete meal nutrients profile, the type of exercises needed to burn the meal calories, and finally data regarding exercises already executed during the day and the food consumed, the system allows the user to better understand the food and exercise correlation and make food and exercise choices better targeting his/her body needs.

### BRIEF DESCRIPTION OF THE DRAWINGS

FIG. 1 contains block diagram of the proposed method.

FIG. 2 shows the block diagram overview of the post-meal blood glucose peak management through exercises.

FIG. 3 details the progress bars for the food, exercise and weight loss progress and goals at a given time of the day.

FIG. 4 shows exercise tracking, as generated by pedometer. Progress bar graphs show calories, steps, distance and active minutes.

FIG. 5 illustrates setting long-term Steps Exercise Goal.

FIG. 6 shows setting long-term Duration Exercise Goal.

FIG. 7 shows long-term Calories Exercise Goal setting. Shown is goal verification in terms of steps and speed needed to burn goal calories.

FIG. 8 shows setting long-term Calories Exercise Goal. Indicated is duration of walking/running needed to burn the goal calories.

FIG. 9 shows verification of the Exercise Step Goal in terms of calories burned in each goal setting.

FIG. 10 illustrated the verification of the Exercise Step Goal in terms of the duration of each goal setting.

FIG. 11 shows the verification of the Exercise Step Goal in terms of distance covered in each goal setting.

FIG. 12 illustrates the verification of the Exercise Duration Goal in terms of calories burned in each goal setting.

FIG. 13 shows the verification of the Exercise Duration Goal in terms of steps covered in each goal setting.

FIG. 14 indicates the verification of the verification of the Exercise Duration Goal in terms of distance covered in each goal setting.

FIG. 15 shows graphical display of the daily exercise tracking with the Calories Goal exceeded.

FIG. 16 gives timed bar graphs illustrating daily progress in steps, exercise calories and distance covered while walking or running.

FIG. 17 summarizes exercise by: total steps, calories, active minutes and average speed. Data for each speed bin is in metric units.

FIG. 18 summarizes exercise by: total steps, calories, active minutes and average speed. Data is presented in imperial units.

FIG. 19 shows the summary of physical activities in terms of total steps, calories, active minutes and average speed from previous days.

FIG. 20 shows daily activities reporting in the selected interval. Reported are: steps, calories, average speed and active minutes.

FIG. 21 shows the progress in Food Calories, Exercise and Metabolism Calories and the Daily Caloric Balance. User-defined goals are superimposed. This is the case when the Daily Caloric Balance is positive.

FIG. 22 shows the bar graphs for Food Calories, Exercise and Metabolism Calories and the Daily Caloric Balance. User-defined goals are superimposed. This is the case when the Daily Caloric Balance is negative.

FIG. 23 shows Short Term Exercise Goals executed once.

FIG. 24 shows Short Term Exercise summary: exercise goal, total steps, calories, duration and average speed. Further, active minutes and steps for all speed bins are reported in user-selected units (metric or imperial).

FIG. 25 shows the daily metabolic, exercise and weight loss calories report. The net food calories to be consumed are presented.

FIG. 26 shows the daily food goal setting per six meals of the day. The doughnut graph gives the calory distribution among all daily meals.

FIG. 27 illustrates setting the carb goals for each of the six daily meals. The doughnut graph gives the distribution among all daily meals.

FIG. 28 shows setting the time of each meal of the day.

FIG. 29 summarizes calories and carbs goals of the given meal and the bar graph progress so far in the day.

FIG. 30 is the same summary of calories and carbs goals from FIG. 29 augmented with bonus calories due to exercise above the daily goal, as well as the imbalance calories consumed in previous meals.

FIG. 31 presents the nutrition information for a food item corresponding to the selected weight, calories, carbs or serving size. The progress graphs indicate the effect of the food item is added to the meal.

FIG. 32 shows the template for entering nutrition information for the food item to be added to the Food Library.

FIG. 33 illustrates selection of a food item by choosing weight, carbs, calories or size, and the corresponding numerical value.

FIG. 34 illustrates the nutrients with emphasis on carb contents. Indicated are carbs reported with and without fiber. The progress graph shows the carbs of the item as added to the menu.

FIG. 35 shows the menu of an indicated meal created so far.

FIG. 36 summarizes nutrients of a meal. The doughnut graph shows the distribution of the meal calories among carbs, proteins and fats.

FIG. 37 illustrates suggested exercises (steps) suggested to expend the food calories and address post-meal blood glucose peak.

FIG. 38 illustrates suggested exercises (duration) suggested to expend the food calories and address post-meal blood glucose peak.

FIG. 39 shows the case when no recommendation for the post-meal exercises is issued.

FIG. 40 describes the exercise in terms of steps needed for burning the total caloric content of a considered food item.

FIG. 41 describes the exercise in terms of duration needed for burning the total caloric content of a considered food item.

FIG. 42 illustrates the summary of the daily trends.

FIG. 43 lists a complete menu of a current day.

FIG. 44 shows bar graphs illustrating the daily consumptions of calories and proteins spread over all daily meals.

FIG. 45 illustrates the contents of fats, carbs and glycemic load of meals of the day.

FIG. 46 displays a doughnut graph of the total daily calories distribution among carbs, proteins and fats.

FIG. 47 shows the weekly summary of calories consumed, exercise calories, step count and active minutes. Their impact on maintaining healthy weight is analyzed using body weight graph in the figure.

FIG. 48 lists the previous week for accessing the daily menu.

FIG. 50 shows a menu of a selected day from the past week.

FIG. 50 includes two bar graphs illustrating exercise and meal calories displayed on the daily basis registered in the last week.

FIG. 51 includes two bar graphs for Daily Caloric Balance and consumed Proteins displayed on the daily basis in the last week.

FIG. 52 displays two bar graphs illustrating the consumption of fats and carbs registered on the daily bases in the last week.

FIG. 53 illustrates the glycemic load of all meals in a day registered in the last week.

FIG. 54 shows the analysis of the progress of the weight-loss program in the last week.

FIG. 55 illustrates the way of setting the new weight-loss plan.

FIG. 56 displays the way to set parameters used to determine among others exercise energy expenditure and step length.

FIG. 57 shows the selection of length, weight and volume units between metric and imperial measurement systems.

FIG. 58 illustrates the way of setting the e-mail addresses of recipients of some of the views of the tool.

FIG. 59 illustrates reporting the total amount of carbs consumed in the product. The used can select carbs with and without fiber.

### DETAILED DESCRIPTION OF THE INVENTION

This invention presents a method and a system for coordinated tracking of the energy expended (exercises and metabolism) and consumed (food) during daily activities. Interleaving energy expenditure and consumption promotes better understanding of user's needs and allows achieving higher fitness level and more appropriate weight management. Further, the nutrient information of a consumed food, paired with precise exercise tracking provides means for better control of postprandial (post meal) blood glucose levels through timed exercises.

Physical activity recording comprises of step count, distance, speed, type of exercise (walk and run), duration and energy consumption. The system applies step length determination based on the user age, gender, weight and height. Based on the motion data, the system determines the step count, movement classification (walk or run), speed and distance estimation and energy consumption [3]. The system can report all exercise and food data in standard (SI) and imperial units.

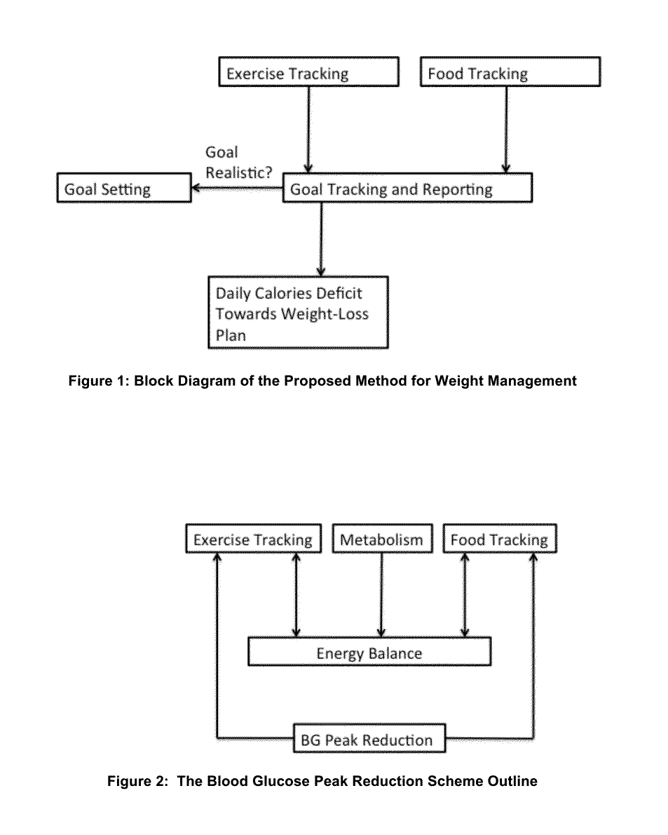

The system allows tracking of daily meals including its caloric value and the constituent nutrients. Food and exercise data is used to track the progress towards the weight loss and various exercise goals. The diagram of the weight-loss progress-tracking scheme is shown in FIG. 1.

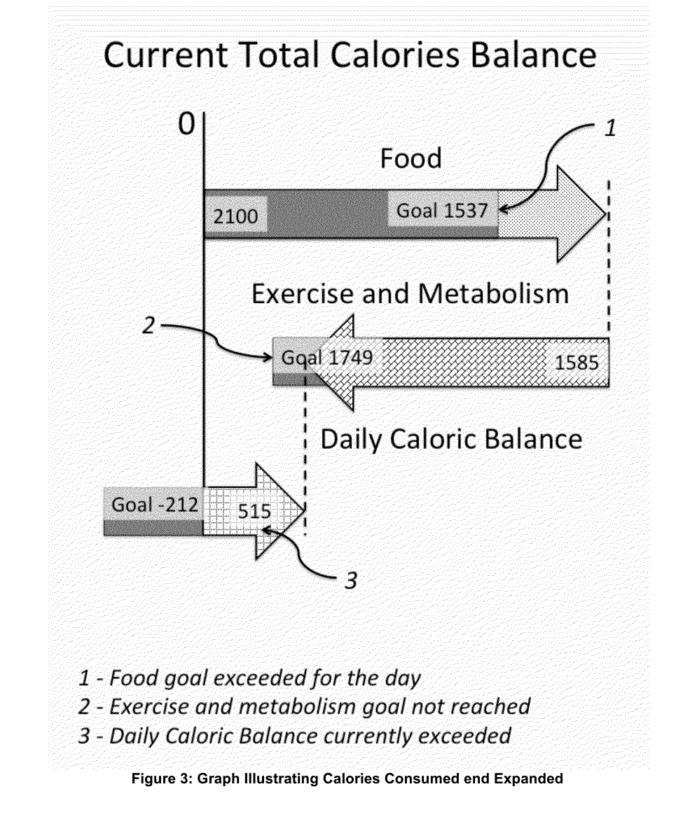

Finally, the proposed system presents the recommended intensity and timing of the exercise relative to the time of food intake with the goal of avoiding blood glucose peaks following the meals. The overall scheme for managing blood glucose peaks is given in FIG. 2.

The proposed invention consists of applying the pedometer in conjunction with the food intake tracking to obtain the timely reporting of the energy balance (incorporating exercise energy expenditure, metabolism energy and food calories), current blood glucose (BG) level, weight loss prospects and the impact of ingested food to the blood glucose of an individual, together with an advice on managing the peak BG level.

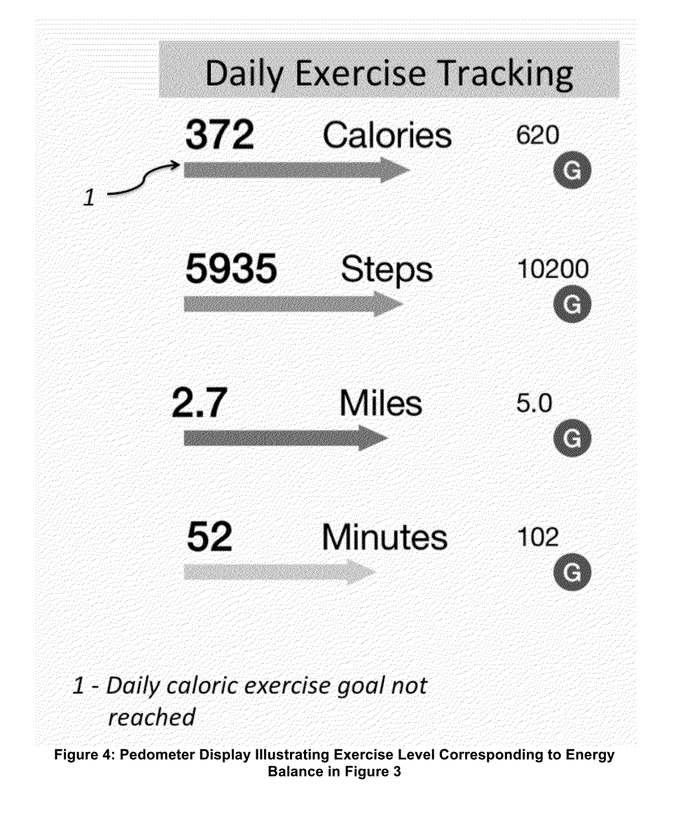

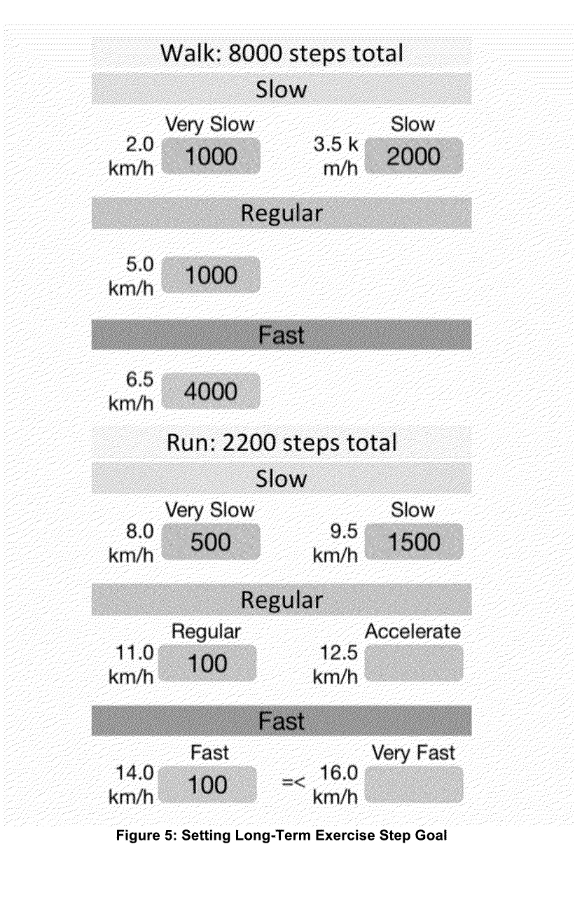

Having a coordinated control over energy expenditure during the daily activities and the energy consumed in food allows the user to better schedule and control the exercises and food intake during the day. This is of particular importance for weight-loss applications [6] and in taking a better control of diseases such as diabetes, where the blood sugar level control is tantamount to the success. FIG. 3 illustrates how interleaving of food calories, exercise and daily caloric balance on one graph display can aid the user in making healthy food and exercise plans for the rest of the day. The Current Total Calories Balance graph, FIG. 3, lays out the case where the calories consumed in food exceed the calories expanded on exercise and metabolism. Further, the Current Total Calories Balance and Daily Exercise tracking graphs, FIG. 3 and FIG. 4 point out that the daily exercise calories goal was not reached yet. Therefore, to bring the Daily Caloric Balance into the goal zone, the user should increase the exercise level and limit the food consumption to the end of the day. More information about the calory consumption reduction is given when entering meal to the system.

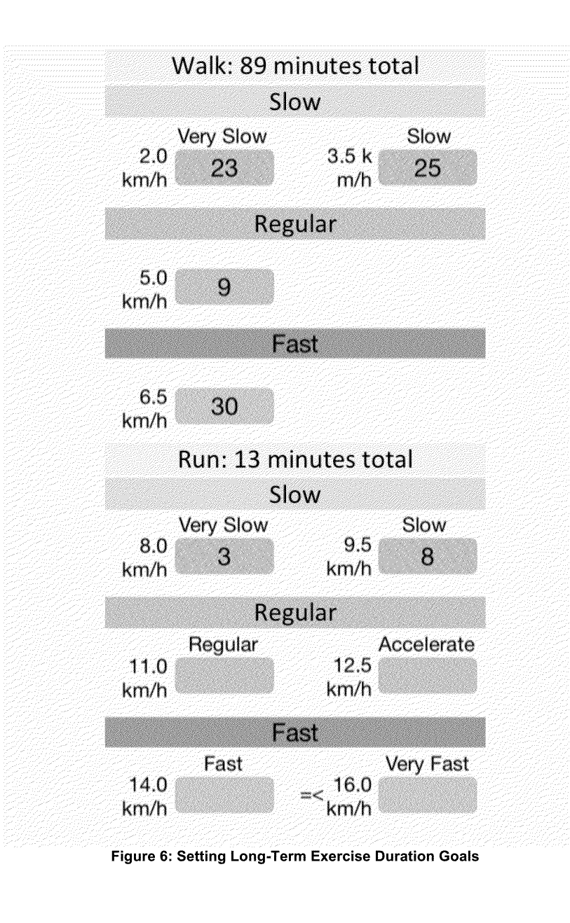

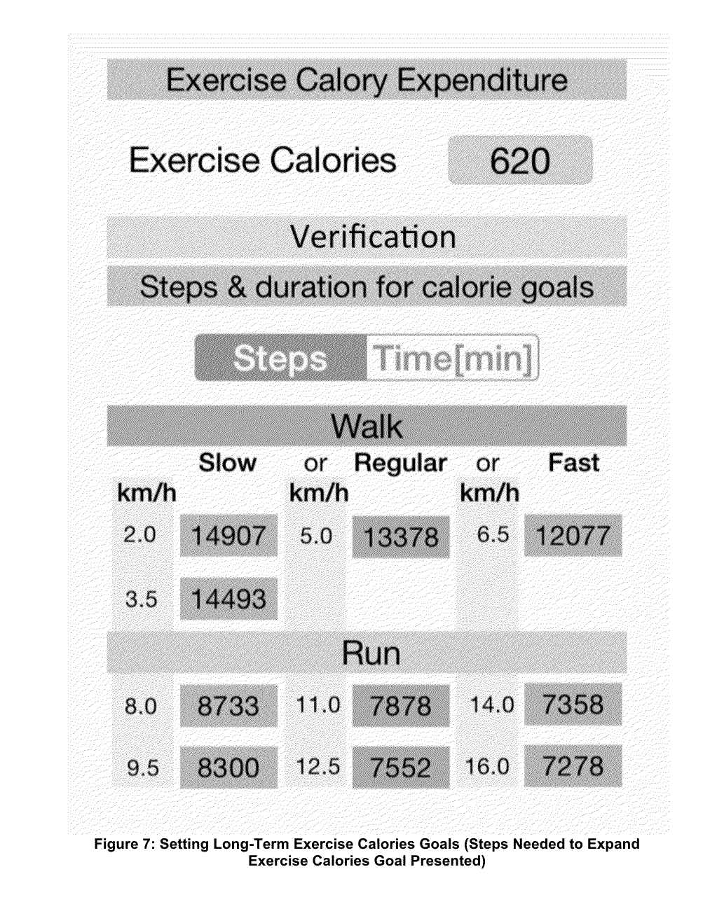

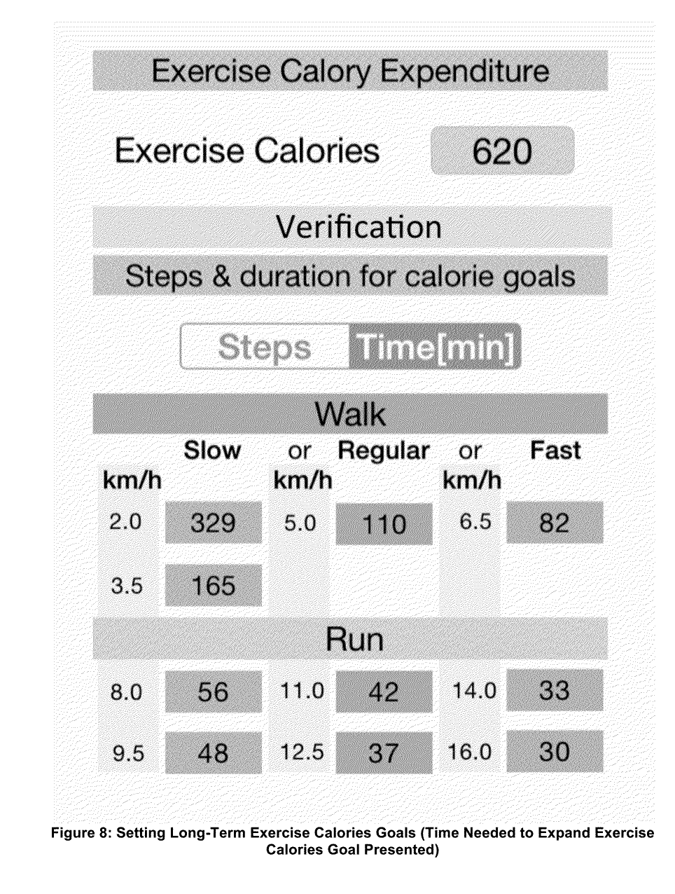

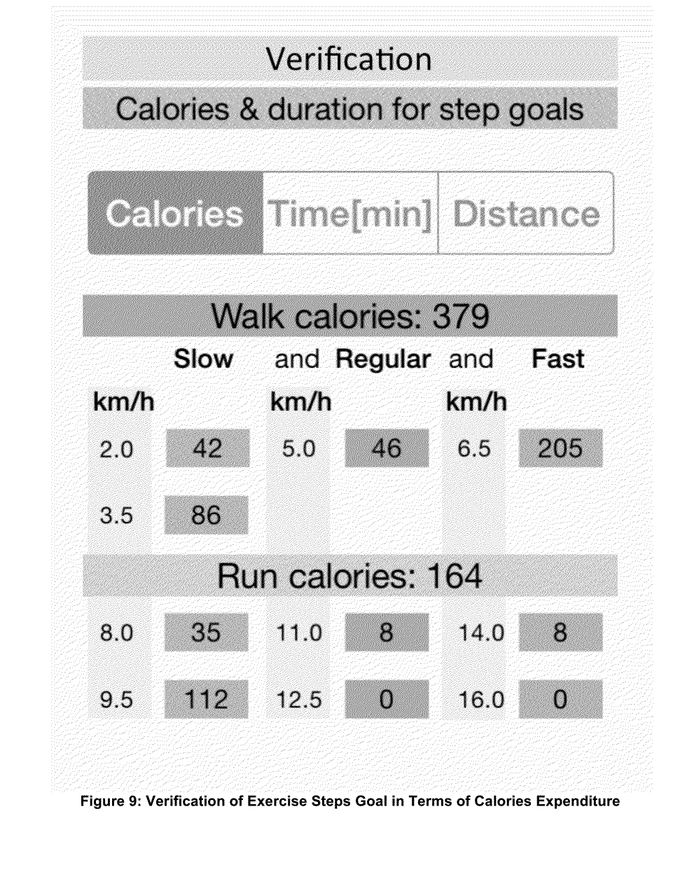

The pedometer is capable of the uninterrupted daily activity tracking, which, in a preferred embodiment, is software on a mobile device, including, but not limited to, smartphones, wearable electronics, portable music players or headphone-based activity trackers. Furthermore, the motion data is in the preferred implementation processed by the pedometer method [3] that allows the user to set a variety of daily exercise goals. In particular, the proposed solution supports the following goals: step count executed with selected speed and type of motion, FIG. 5; duration of walking and running with different speeds, FIG. 6; or energy expenditure (exercise caloric balance), FIG. 7 and FIG. 8. The goal can be also set as a composition of shorter terms sub-goals to achieve in given time intervals.

Goals—Exercise.

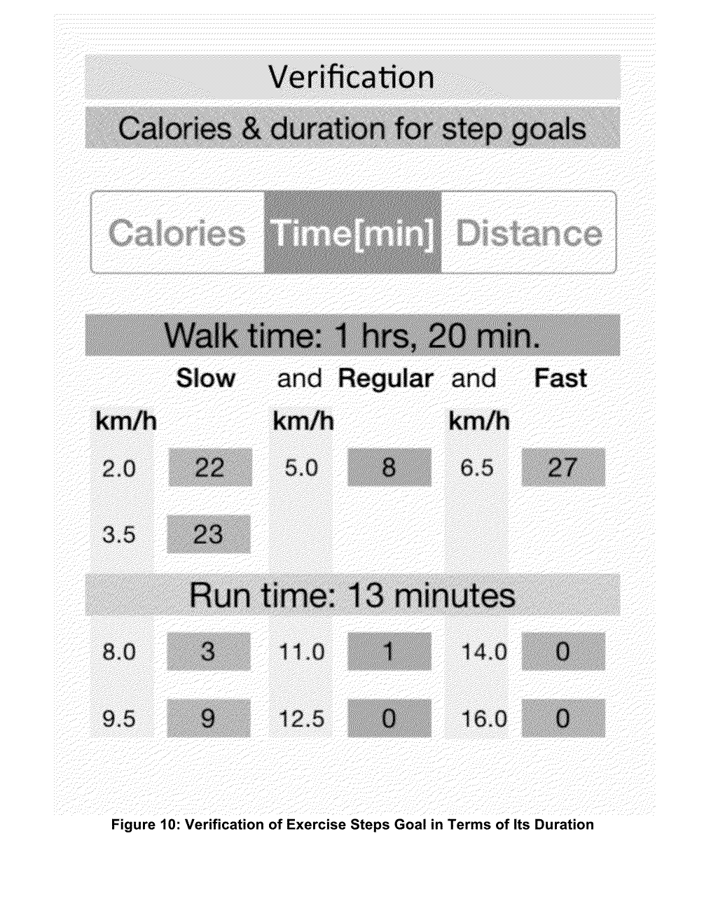

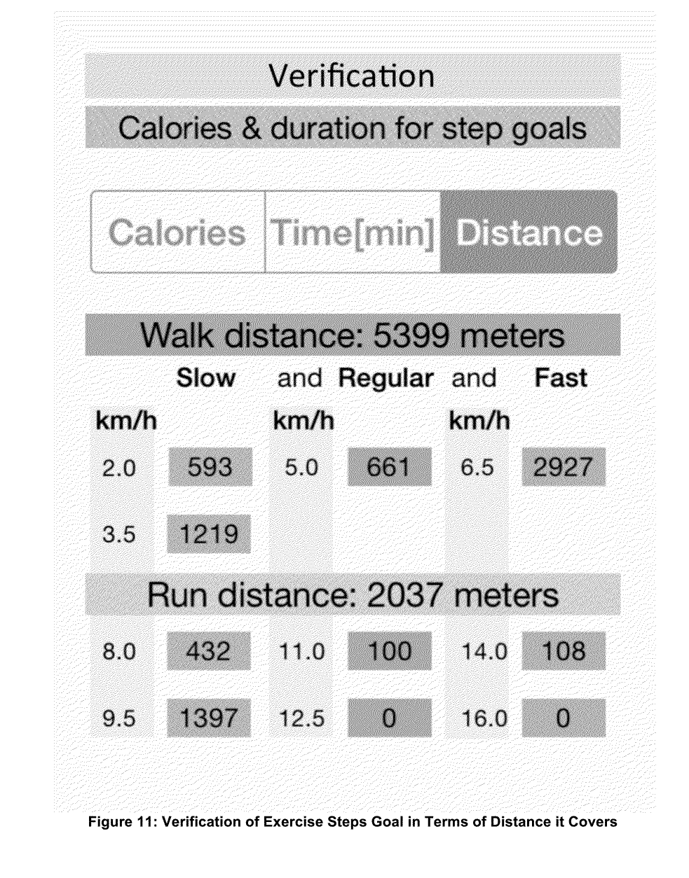

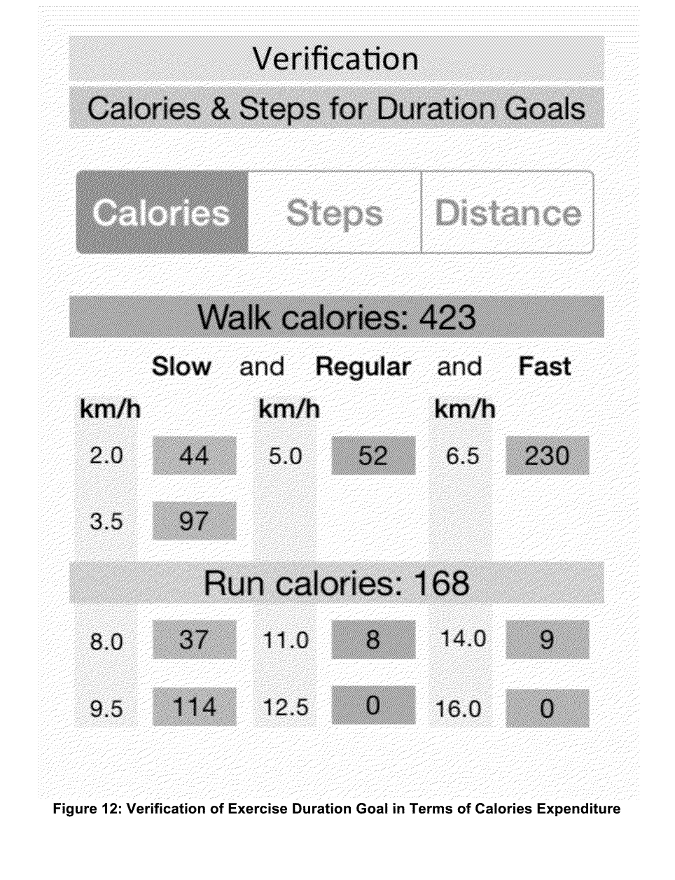

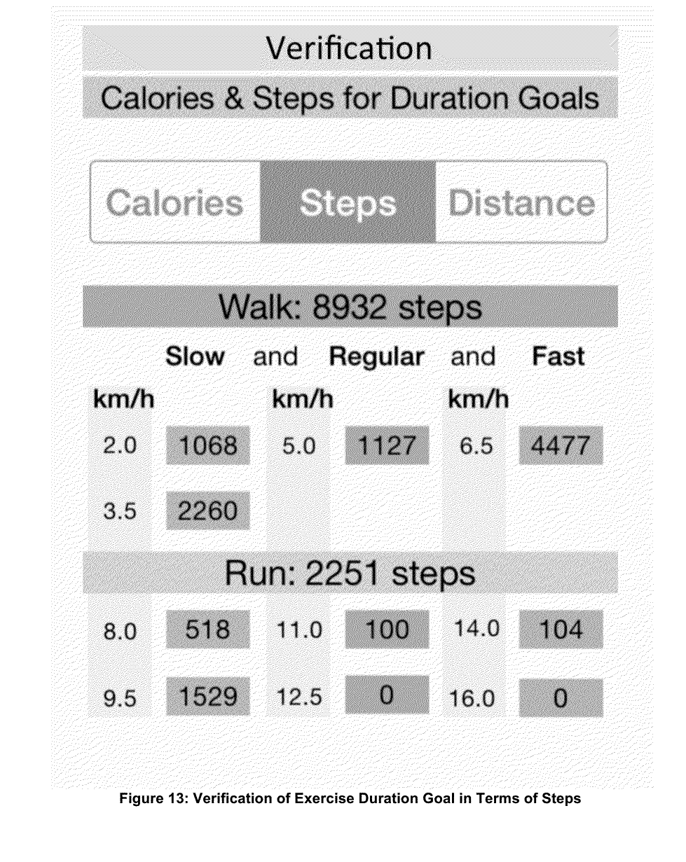

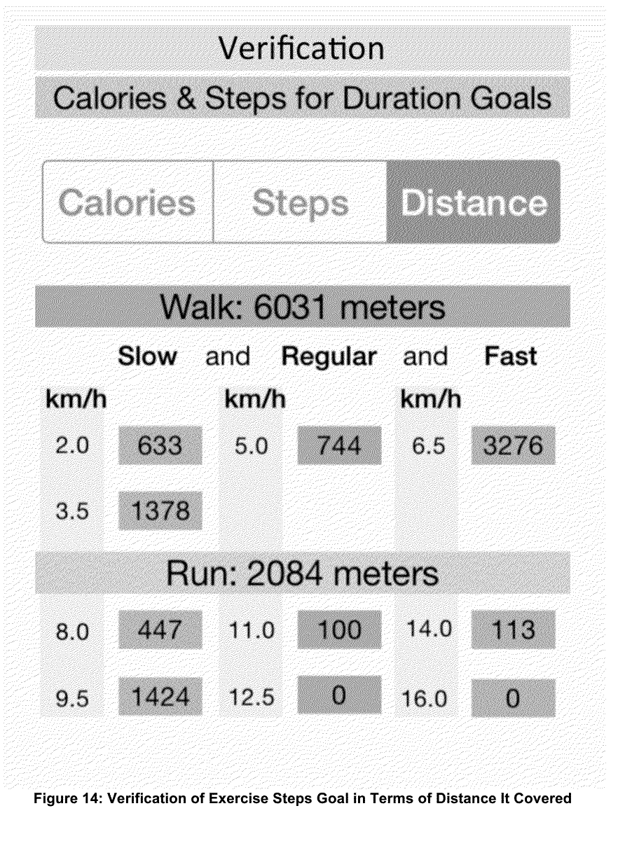

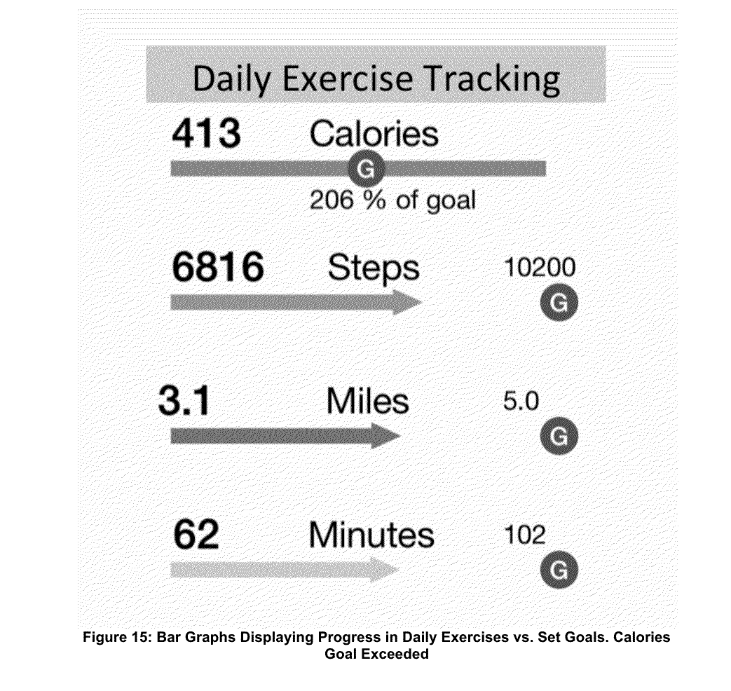

The goals can be set in terms of total amount of steps committed during the day, exercise duration (active minutes) or in terms of total exercise calories. The goals are exchangeable, i.e., setting one goal will result in pre-setting this goal in terms of two remaining goals. The detailed options of each of the exercise goals are as follows:
Step Count. Two types of motion are walk and run.
   The corresponding step goals for walking can be set in speeds: slow (1 and 2 mph), regular (3 mph) and fast (4 mph). Corresponding types of walk in metric system are: slow (2.0 and 3.5 km/h), regular (5.0 km/h) and fast (6.5 km/h), FIG. 5
   Run steps can be set in: slow (5 and 6 mph), regular (7 and 8 mph) and fast (9 and 10 or greater mph). Corresponding types in metric system are: slow (8.0 and 9.5 km/h), regular (11.0 and 12.5 km/h) and fast (14.0 and 16.0 or more km/h), FIG. 5.
   Multiple selections of speeds in walking and running allow tracking activities consisting of different levels of intensity, e.g., 15 minutes of slow running and 20 minutes of fast walking.
   Step Count goal can be verified in terms of calories, duration and distance.
      Calories verification—the system informs the user about the energy expenditure of walking and running with each of the selected speeds, FIG. 9;
      Duration verification—provides the duration of walking and running in each of selected speed options, FIG. 10;
      Distance verification—the system calculates the distance covered by walking and running with each of the selected speeds, FIG. 11.
Exercise Duration. Users can select the duration of walking or running in ten different speeds.
   The duration goals for walking can be set in: slow (1 and 2 mph), regular (3 mph) and fast (4 mph) speed. Alternatively, the corresponding types of walk in metric system are: slow (2.0 and 3.5 km/h), regular (5.0 km/h) and fast (6.5 km/h), FIG. 6.
   The duration of run can be set in the following six speeds: slow (5 and 6 mph), regular (7 and 8 mph) and fast (9 and 10 or greater mph). Alternatively, the corresponding types of run in metric system are: slow (8.0 and 9.5 km/h), regular (11.0 and 12.5 km/h) and fast (14.0 and 16.0 or greater km/h), FIG. 6.
   Multiple selections of speeds in walking and running are allowed.
   Exercise Duration goal can be verified in terms of calories, steps and distance, FIG. 6.
      Calories verification—the system informs the user about the energy expenditure of walking and running with each of the selected speeds, FIG. 12;
      Steps verification—the system estimates the step count of walking and running in each of selected speed options, FIG. 13;
      Distance verification—the system approximates the distance covered by walking and running with each of the selected speeds, FIG. 14.
Exercise Calories. The third exercise goal options is Calories Goal, which allows the user to set the daily total amount of calories, which he/she wants to expand on exercises.

The calories goal can be verified in terms of steps and duration.
Steps verification—the system informs the user about the step count in each of ten speeds of walking and running, which execution is equivalent to the set caloric goal, FIG. 7;
Duration verification—the system lists the duration of walking and running in each of ten speeds, which execution is equivalent to the set caloric goal, FIG. 8.

The exercise goal is reached either when the user-set goal plan is realized, or when the energy expenditure of the goal algorithmically determined is met.

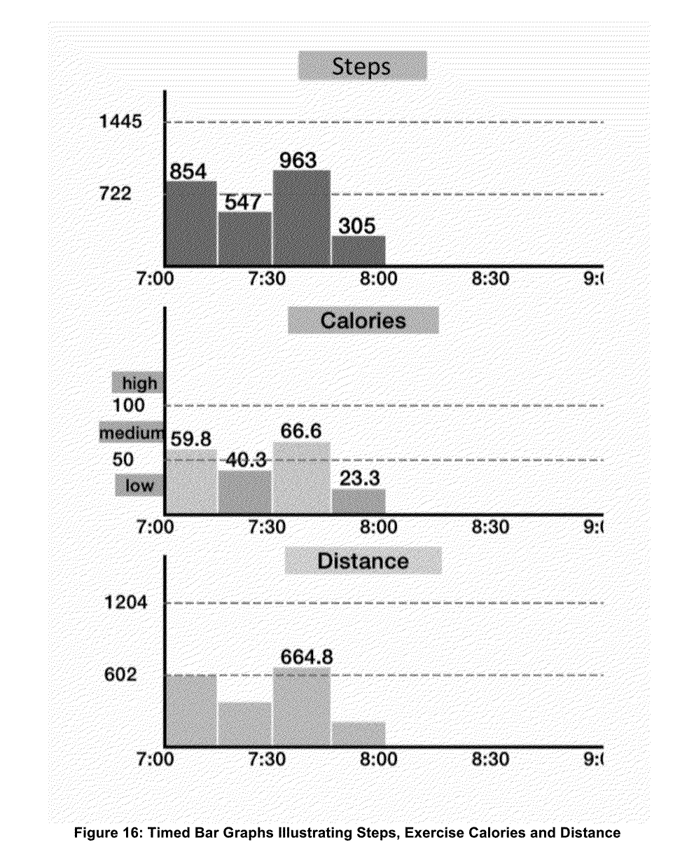

Pedometer tracking results are displayed in a separate view. Starting from the midnight of the current day, individual progress bars present: calories expended during all daily activities, cumulative step count, distance and active minutes. Each progress bar is scaled to the analogous exercise goals set by the user (calories, steps, distance and active minutes), FIG. 4. The progress bar does not saturate, but shows the values relative to the goals even as the goals are exceeded, FIG. 15.

Detailed Exercise Reporting—

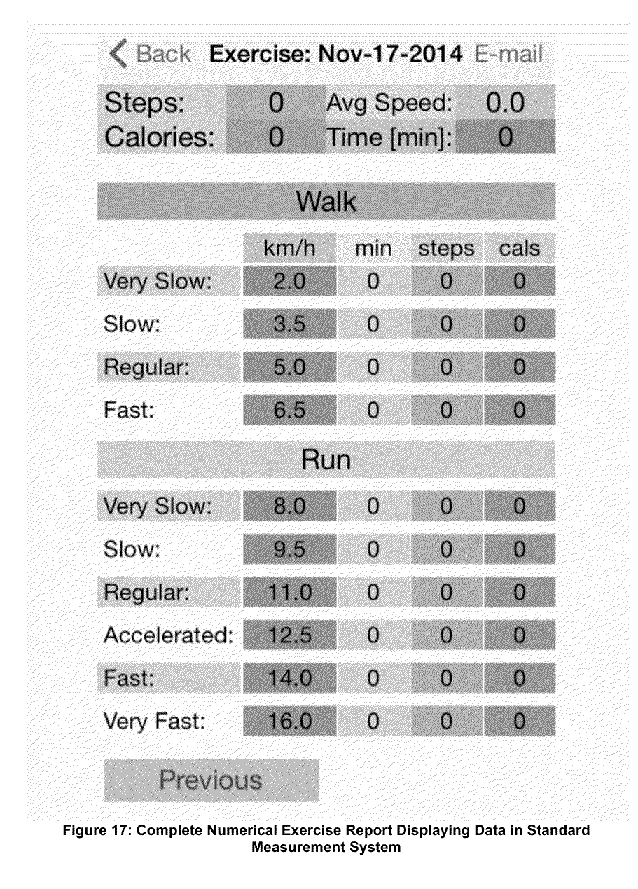

Bar Graph. Reporting of steps, energy expenditure and covered distance is represented in one preferred implementation by a bar graph. Each of the above values is displayed on the daily scale. The numerical profiles of the three above exercise parameters are placed over the corresponding bars, FIG. 16.

Detailed Exercise Reporting—

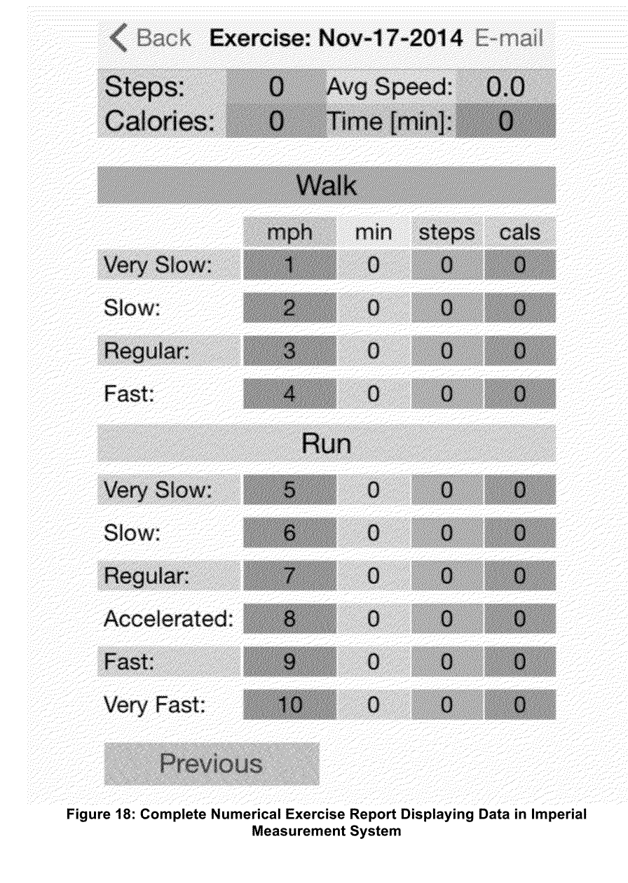

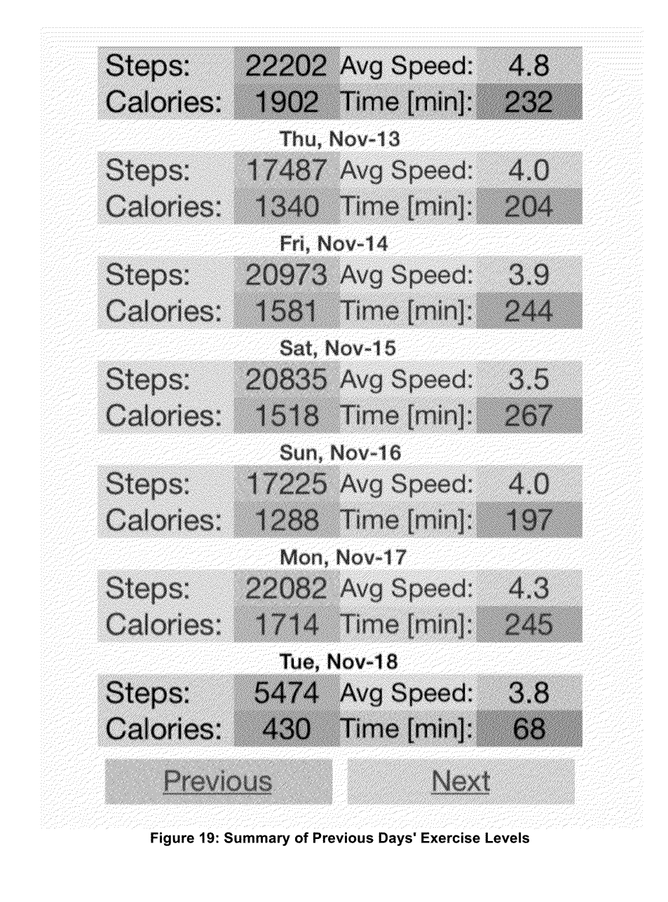

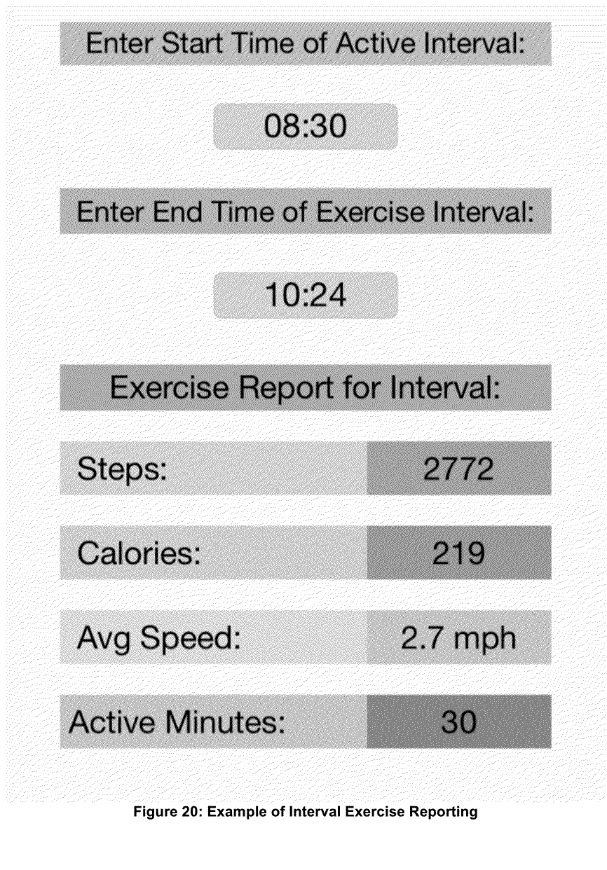

Numerical. An alternative tabular display summarizes the current daily physical activities by showing:
Total steps, average speed of moving, total calories expended on exercises and active time;
Detailed analysis of walking and running in ten speed bins of walking and running. The ranges of speeds for walking in standard system are: 2.0 km/h (very slow walk), 3.5 km/h (slow walk), 5.0 km/h (regular walk) and 6.5 km/h (fast walk). The ranges of speeds for running in standard system are: 8.0 km/h (very slow run), 9.5 km/h (slow run), 11.0 km/h (regular run), 12.5 km/h (accelerated run), 14.0 km/h (fast run) and 16.0 km/h (very fast), FIG. 17. Alternatively, the speeds for walking in standard system are: 1 mph (very slow walk), 2 mph (slow walk), 3 mph (regular walk) and 4 mph (fast walk). The ranges of speeds for running in standard system are: 5 mph (very slow run), 6 mph (slow run), 7 mph (regular run), 8 mph (accelerated run) 9 mph (fast run) and 10 mph (very fast), FIG. 18.
   For each speed bin displayed are: active minutes, total number of steps and calories expended, FIG. 17 and FIG. 18.
      Further, previous days' physical activities (steps, average speed, active time and calories expended) are accessible from this view, FIG. 19.

Detailed Exercise Reporting—

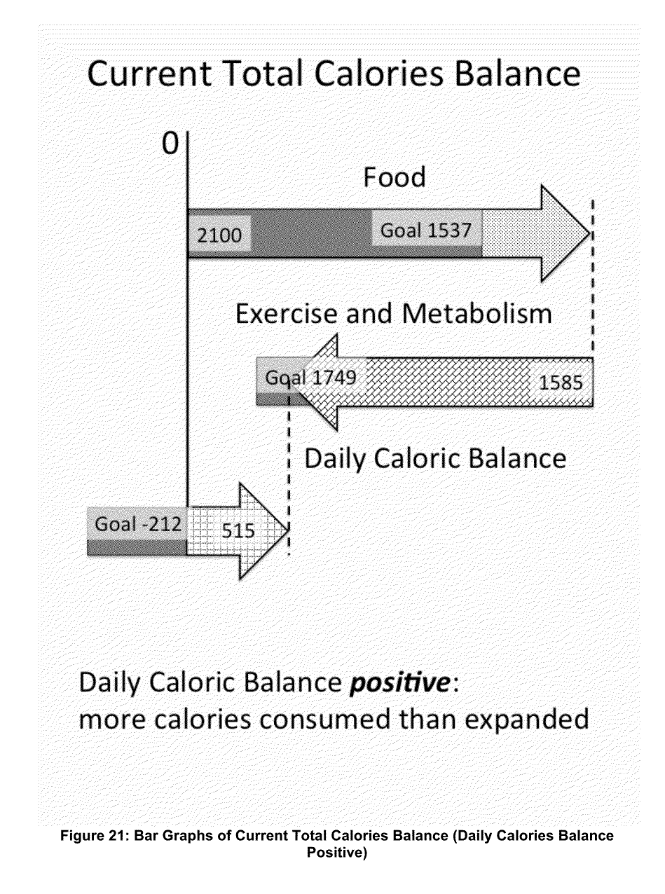

Intervals. Physical activities exhibit significant impact on dosing medications in diseases such as diabetes. To better understand the impact of exercises on diabetes patients, the tool provides a report in the user-selected time interval, FIG. 20.

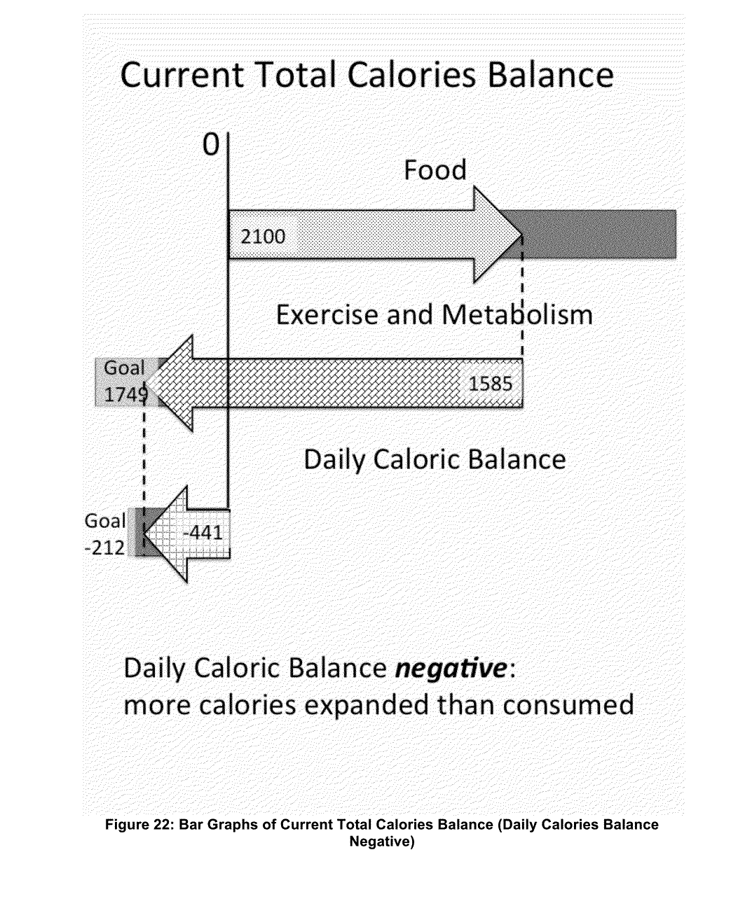

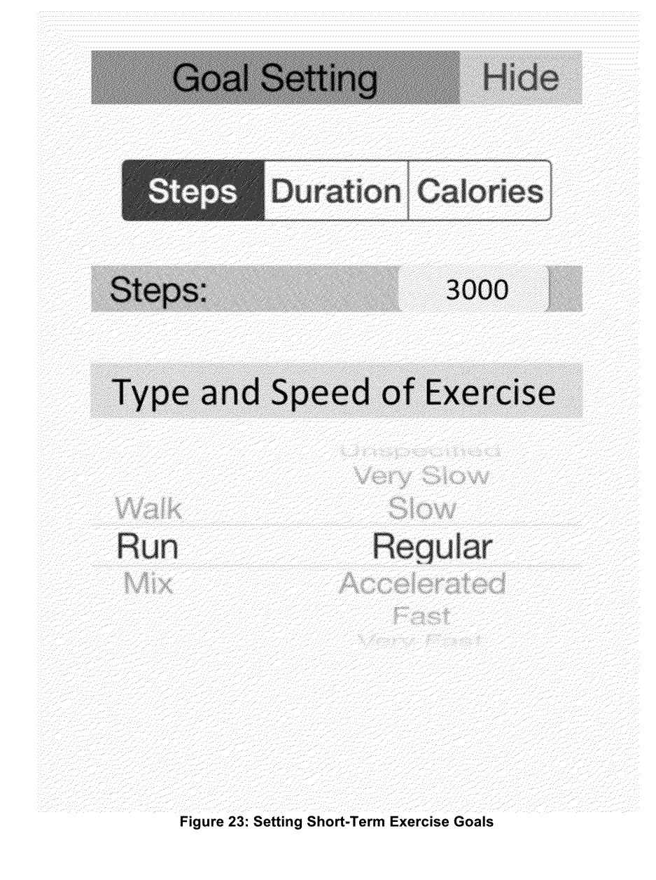

In addition to showing the daily fitness level, the pedometer data is also incorporated for better understanding of current energy balance. Here, energy balance is given as the difference between calories consumed (food) and energy expended (exercise and metabolism), FIG. 21 and FIG. 22.

Energy balance monitoring starts daily at midnight, and progresses through the day. The reporting of current energy balance is done in real time on a single progress bar graph, FIG. 21 and FIG. 22.

Superimposed to the daily energy balances are the daily goals for energy expended, energy ingested and the weight loss. In the preferred implementation, the values are compared graphically to goal bars. Goal values are listed inside the bar, FIG. 21 and FIG. 22.

Daily energy balances are in one instance reported as linear progress bars where the energy expended is subtracted from the energy consumed, resulting in the balance that contributes to the weight loss or weight gain, depending on the sign of the balance, FIG. 21 and FIG. 22.

Short Term Exercise Goals.

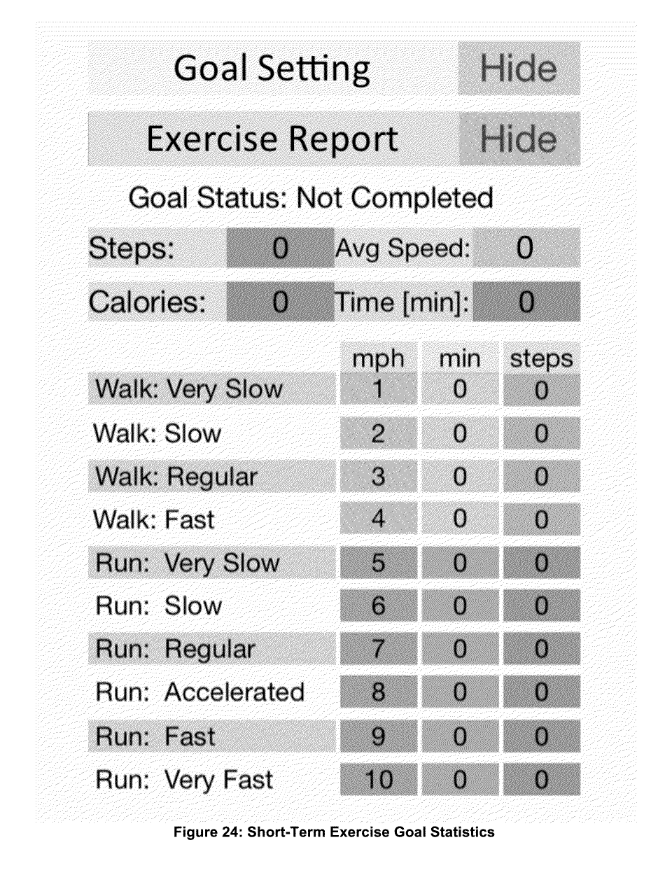

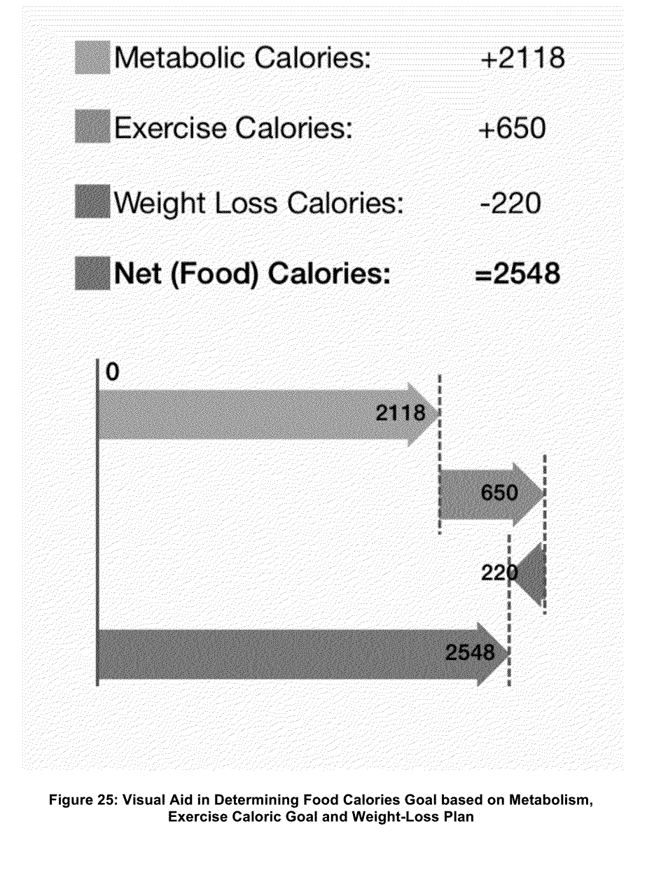

In addition to long-term exercise goals, which stay active day after day, users can set a short-term exercise goal. Similarly to long-term goals, users can select the goals for steps, duration or calories. For steps and duration goals, the user can select the type of exercise (walking, running or mixed) and the speed (unspecified, very slow, slow, regular, accelerated, fast and very fast), FIG. 23. The speeds of exercise correspond to numerical values associated with these names, and described above in paragraph [0087]. The user can verify the statistics of the exercise any time during and after the routine, FIG. 24.

Meal.

The food entry is organized by daily meals, such as breakfast, morning snack, lunch, afternoon snack, dinner and an extra meal. A meal is entered by selecting a set of the specific meal items by users. To facilitate more appropriate food item's selection, users can consult the pre-set meal goals such as calories per meal and carbs per meal.

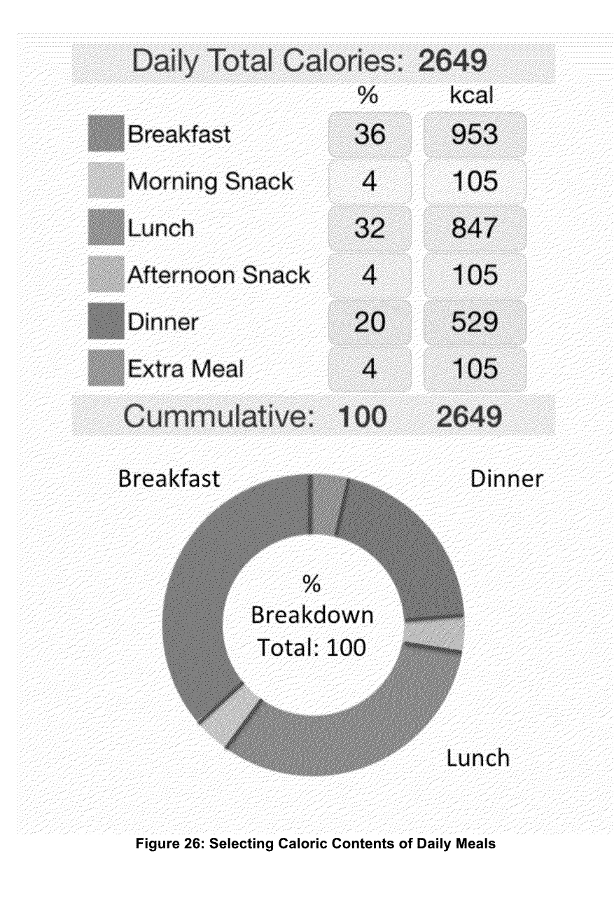

Total daily food calories are determined and suggested to the user. The calories to be consumed is a function of the user's metabolism, exercise caloric goal, and, if set, weight-loss calories balance. Relation between metabolic, exercise, weight-loss and food calories is illustrated using the graph bar, FIG. 25.

Calories Percentage Planner.

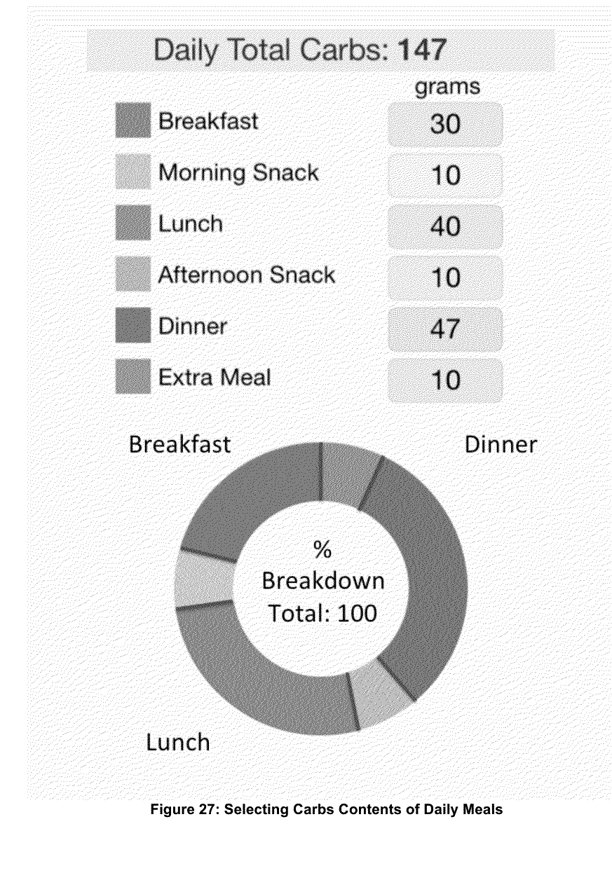

Given the calories to be consumed throughout the day, users can specify the amount of calories allotted to each meal: in breakfast, morning snack, lunch, afternoon snack, dinner and extra meal. Given total food calories goal, users can enter meal entries either in absolute values, or in percentages of daily values, FIG. 26.

The correctness of recorded data is verified numerically by checking that all meal calories ad up to food calories goal and all meal percentage calories assignments add up to 100%. Further, to visualize the calories distribution among daily meals the doughnut graph is used to show the meals' calories split, FIG. 26.

Daily Carbs Meal Planner.

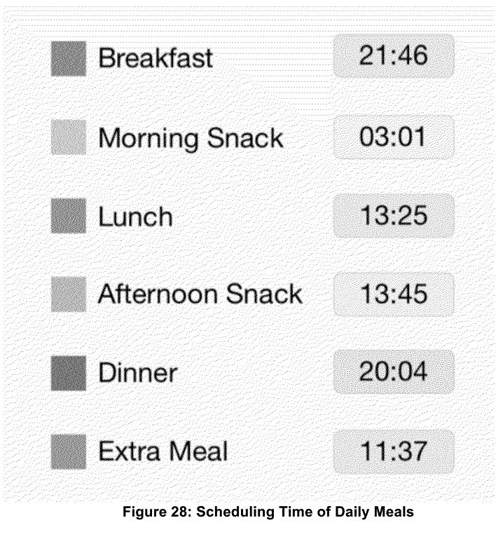

Unlike the food calories, the overall daily carbs goal is not a derivative of metabolism, exercise and weight-loss calories planning, but depends on personal meal habits as well as possible health issues such as diabetes. Therefore, the carb amount, as well as the daily distribution, is determined by the user. The process of assigning carbs goal for each daily meal is presented in FIG. 27. The doughnut graph, FIG. 27, showing the percentage distribution of carbs goals among all daily meals helps user to make balanced meal choices.

Daily Meal Timing Habits.

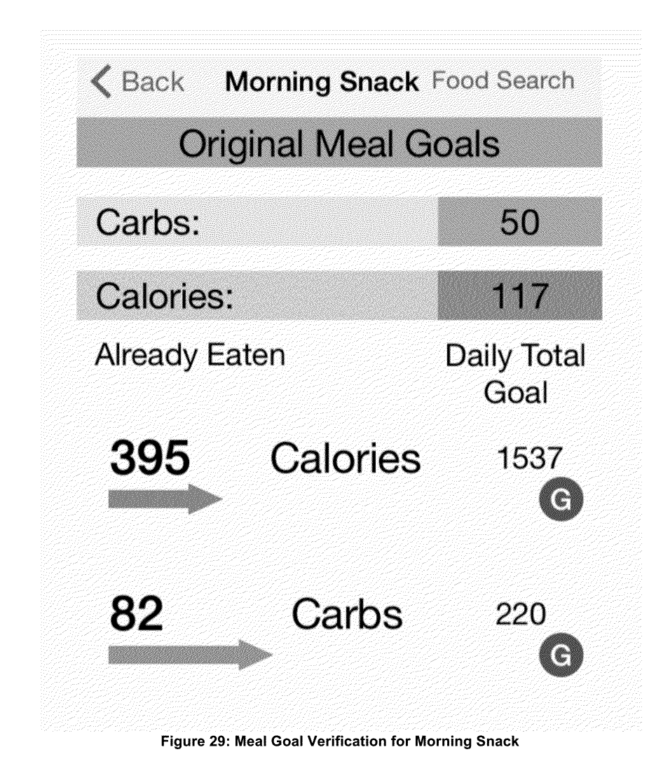

Keeping track of the consumed food throughout the day is important for better coordination of exercises and meals, and, hence, the correctness of determining daily calories balance. Daily Meal Timing entry is created to reminding the user about the need of entering each meal data. Users specify the time of each meal of the day based on his/her habits or constraints, FIG. 28. If at the specified time of a meal, the meal user did not enter the food contents of a meal scheduled at that time, a reminder will be issued.

Tracking Calories Consumption.

As the time progresses, the food taken is compared against the amount scheduled in the daily timing and meal goals. Tracking food calories is then reported by means of two overlapped progress bars. On the top is the bar with an arrow that displays the current amount of calories ingested, while underlying that progress bar is a greyed bar displaying current food energy goal, FIG. 21 and FIG. 22.

Creating a Meal.

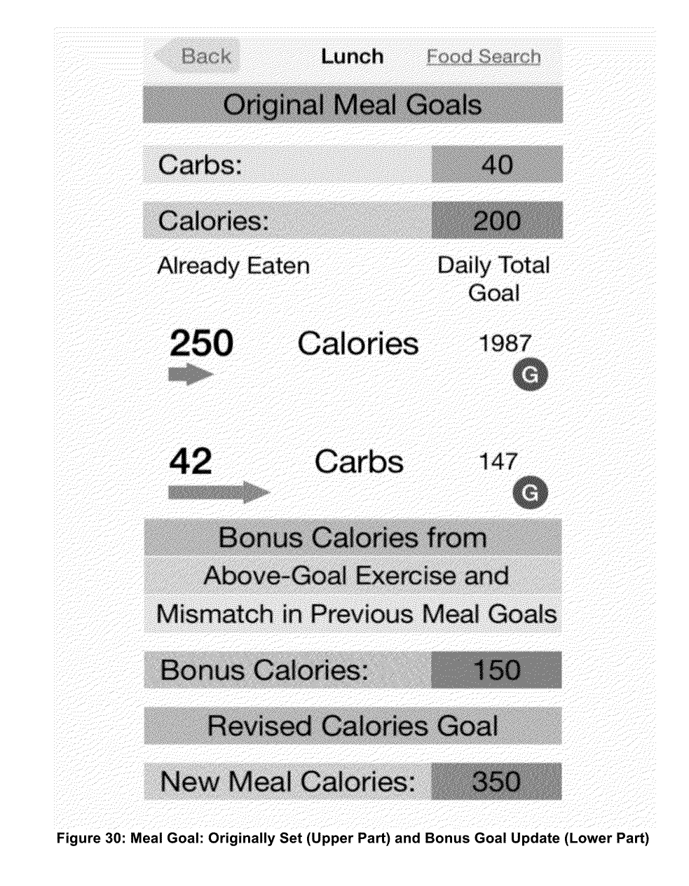

Each time the menu for the given meal is created, a user is presented with calories and carbs goals set for this particular meal in Goal section. Along with listing the calories and carbs goals, the user is also informed how many calories and carbs were consumed so far in the day. The meal goals, established in the Goals sections, can be adopted without any adjustments to guide the food intake throughout the day, FIG. 29. Alternatively, an option is provided to adjust the food intake based on the current exercise performance and food consumed.

Exercise Factor (Exercise_Factor) in Meal Goal Update.

As the system traces in real time energy expenditure in exercises, at the time of the each meal selection, the system knows whether the exercise caloric goal set in Goals was already reached and exceeded. If the exercise goal was not reached at the time of the current meal, then the exercise factor is not considered in the update of the original goal of the current meal. If however, the exercise goal was exceeded at the time of the current meal creation, then the total exercise caloric expenditure is considered at the time of the preceding meal. Given that, following two steps are undertaken by the tool in order to calculate the caloric surplus of the exercise:
1. If the exercise caloric goal was not exceeded at the preceding meal, then the extra exercise calories at the time of the current meal are calculated as the difference between the total energy calories at the present meal and the exercise goal calories for the day.
2. If the exercise caloric goal was exceeded at the time of the previous meal, then the surplus of the bonus calories coming from over the goal exercises at time of the current meal is equal to the current total exercise calories minus the total exercise calories at the previous meal.
   Extra calories coming from exercises constitute one component of the bonus calories, which can be used to upgrade the calories goal of the current meal.

Food Factor (Food_Factor) in Meal Goal Upgrade.

The system analyzes the caloric contents of the food consumed in previous meals, and compares it to the previous meals' goals. The food factor of the caloric bonus update of the is calculated as a difference between the total amount of calories consumed so far exceeds the sum of all updated caloric goals of all meals preceding the currently considered meal goal. The positive value of the above difference indicates that the calories consumed in all the previous meals exceed the sum of updated caloric goals of all the previous meals. Hence, the amount of calories designated for the current meal should be reduced. Otherwise, if the above difference is negative, calories consumed were below the sum of the updated caloric goals of all the previous meals. This leaves some room for extra calories in the current meal.

The previous meals goals used in the comparison are updated in the following way:
1. The breakfast caloric goal can be increased by the excess exercise calories if the daily exercise caloric goal is exceeded before breakfast. The increase is calculated as a difference between the current value of calories exerted in exercises and the daily exercise caloric goal. The breakfast updated goal then becomes:

Breakfast_updated_goal=Breakfast_original_goal+Breakfast Exercise factor.
2. The food factor for the caloric goal update of the meal immediately following breakfast, i.e., Morning Snack is calculated by subtracting the updated breakfast goal from the total calories consumed at breakfast.

Breakfast_Food_factor=Food_so_far−(Breakfast_updated_goal),
where_Food_so_far denotes the total amount of calories consumed so far. In this case, it would be the total calories consumed for Breakfast.
Then, the updated caloric goal of Morning Snack is:

Morning_Snack_updated_goal=Morning_Snack_original_goal+Morning_Snack_Exercise_factor−Morning_Snack_Food_factor,
3. The updated goal of Lunch, which is following Morning Snack is:

Food_factor=Food_so_far−(Breakfast_updated_goal+Morning_Snack_updated_goal),

Lunch_updated_goal=Lunch_original_goal+Lunch_Exercise_factor−Lunch_Food_factor,
4. The remaining meals are updated in the similar manner as Morning Snack and Lunch, with the difference being the incorporation in the food factor the additional meal goals preceding the considered meal.

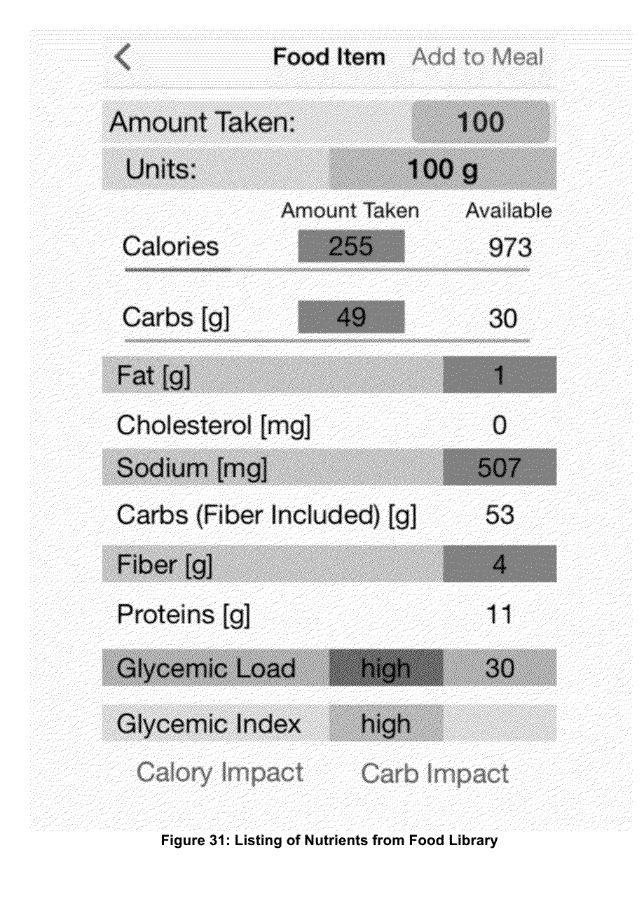

The user is given the original food goal and the bonus calories, together with the new meal goal obtained by updating the original meal goal with bonus calories, FIG. 30. The user has a choice of selecting either the new, daily updated meal goal, or to use the original meal goal. For example, the user can treat above goal exercise calories as additional calories to meals, or put them towards weight-loss plan.

Carbs goal and it daily alterations, as well as the carbs monitoring cannot be adjusted the way the caloric goal is tracked and modified during the day. The goal for carbs consumption per meal depends on many factors. For example, users with diabetes generally have some well-defined carb plan. However, if carbs consumption in one meal does not adhere to the set goal, the carbs intake in other meals is generally not modified, but follows the previously set scheme.

Food items for meal creation can be selected from Food Library comprising over 3600 food items from major food groups such as: bakery (muffins, rolls, buns, croissants), breads, beverages (alcoholic and non-alcoholic), cakes, cereals, cookies, crackers, candies, cold-cut-meads, dairy, eggs, fats, fish, fruits, grains, meats, nuts and seeds, pasta, vegetables, yogurts (including brands names, frozen yogurts and ice-creams).

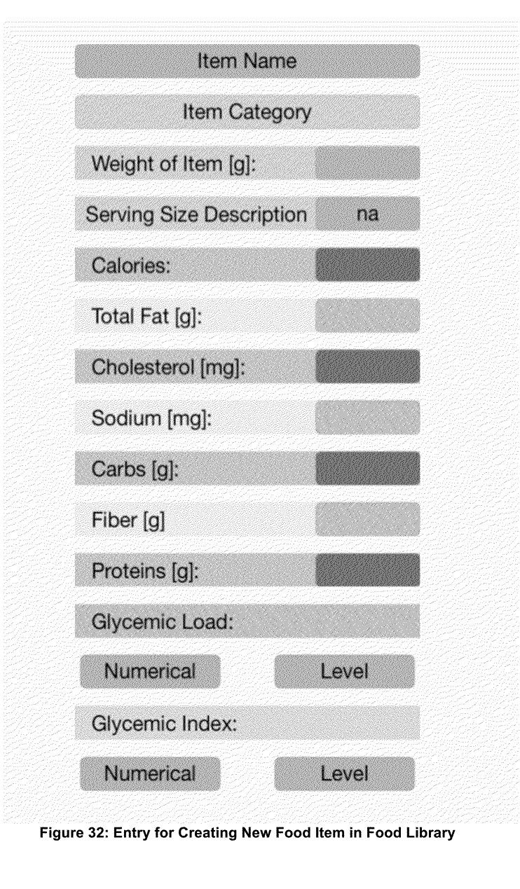

Each food item has nutrients listed per selected product weight, carbs, calories or serving size (for pre-packed products). The product nutrients will be listed for the weight, calories, carbs of serving size selected by the user. In addition to calories, carbs, total fat, cholesterol, sodium, fiber and proteins, each food item has listed glycemic index and loads, FIG. 31.

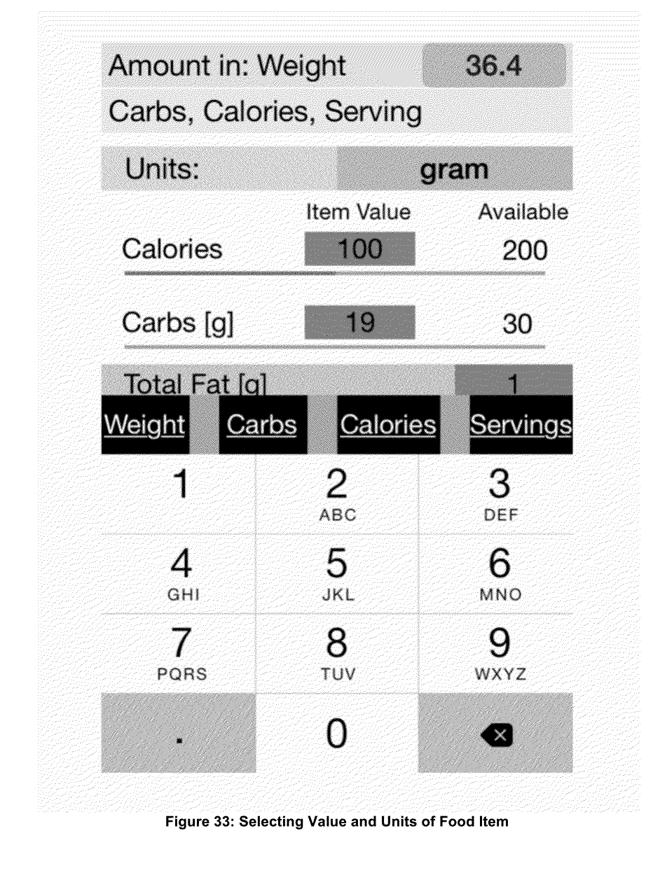

If a food item is not available in Food Library, the user can create a new entry in Food Library and deposit all the nutrients of the not listed food item, FIG. 32.

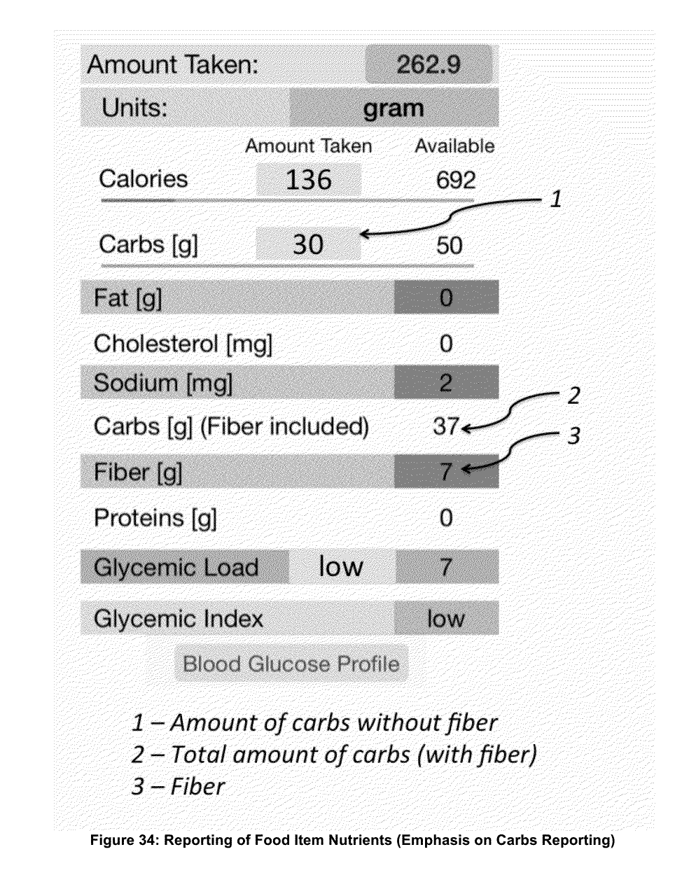

Users can choose the size of a food item by selecting: weight, carbs, calories or serving size, FIG. 33. For example, if the user selects the weight of the food item to be 120 g, then all the nutrients of this food item will be reported for 120 g. Further, if the user chooses to consumed 200 calories of a given food item, then the tool will calculate the weight corresponding to 200 calories, and will report all the remaining nutrients for that weight. Similar procedure is repeated if the user selects the carb content of the food item. Selection of calories of the food item is particularly useful for people on weight-loss program, while the option for carbs selection is of importance for users with diabetes, who create their meals based on the scale carbs selection method.

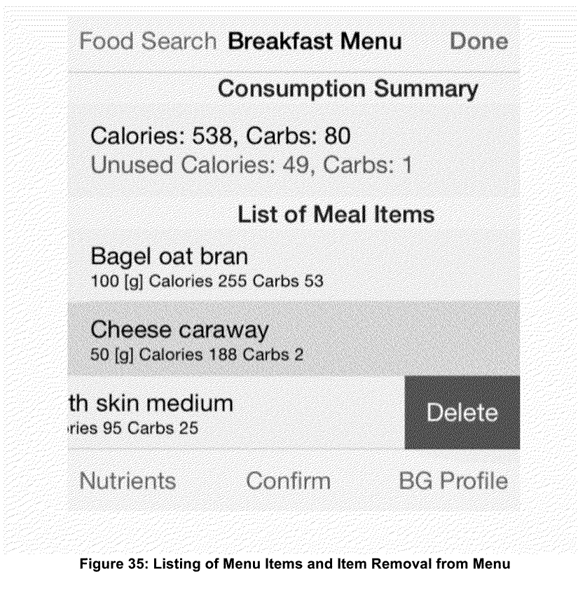

In listing of the food item nutrients, the user is given the total amount of carbs including fiber. However, some diets require that the reported amount of carbs consumed in a meal does not include fiber. Therefore, the user has an option to either include the reporting of fiber in the amount of carbs consumed, or exclude fiber in the reporting. For example, FIG. 34 illustrates the case when fiber is excluded from the notification of consumed carbs. In FIG. 34, the tool shows that 30 g of carbs was consumed. However, for completeness, the entry Carbs [g] (Fiber Included) reports the total amount of carbs in the food item including 7 g of fiber. As users with diabetes generally do not include fiber in the carbs calculation, the reported amount of carbs consumed (total carbs—fiber) can be then used to decide, for example, about the amount of insulin needed to digest these carbs.

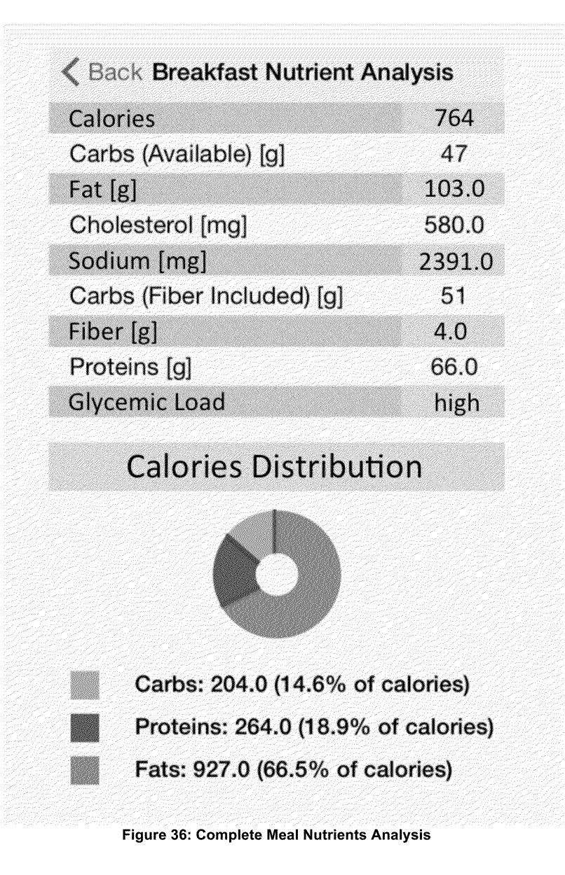

After adding an item to the menu, users can verify the cumulative contents of nutrients of the meal created so far. Each food item added to a given meal can be also removed from the meal by the user, FIG. 35.

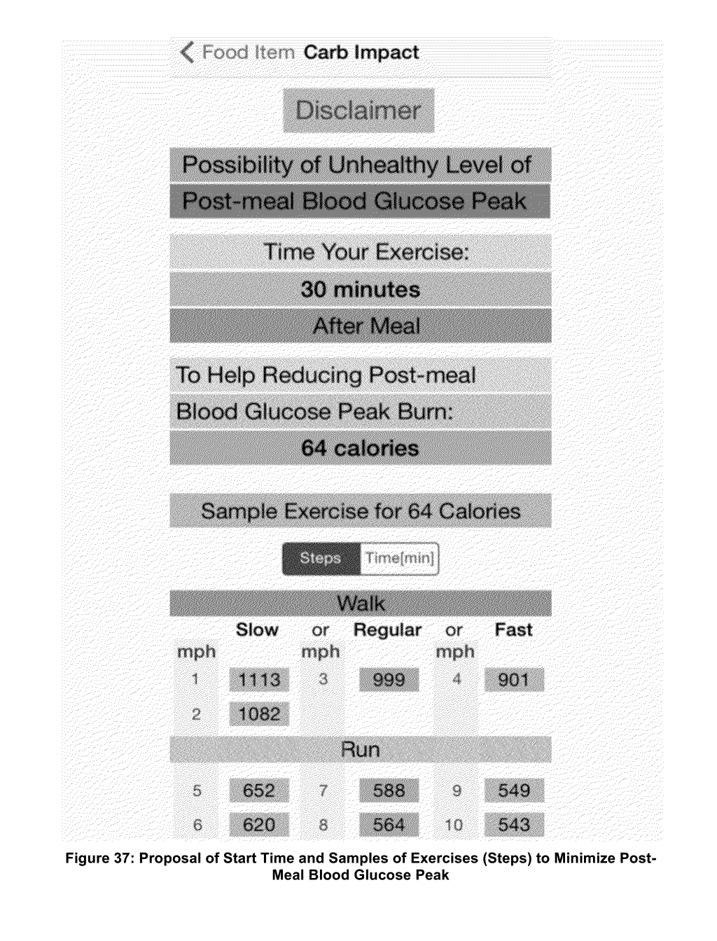

Upon completing the menu for a given meal, the user can verify the nutrients of the meal. This includes reporting of: calories, carbs, fat, cholesterol, sodium, fiber, proteins and glycemic load. Further, the tool provides the calories distribution among carbs, proteins and fats, FIG. 36.

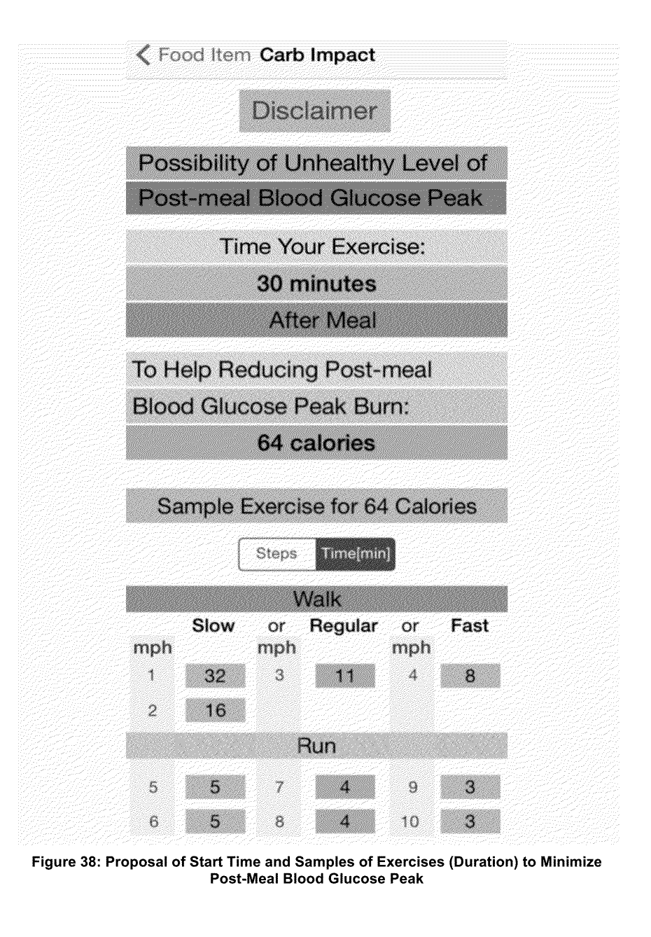

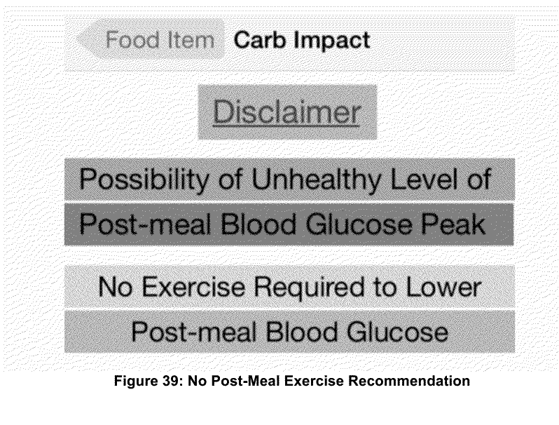

For every food item in Food Library, as well as for every complete meal entered, the presented system obtains the glycemic index and load. Based on the glycemic index, load, and remaining nutrients profile of the meal, the system produces gives estimation whether the post-meal blood glucose (BG) peak would reach an unhealthy level. If the system anticipates that consumed carbs have such an impact, then it issues a recommendation for an exercise that expends the amount of calories helping to bring the post-meal blood glucose peak to a healthy level. Further, an exercise endorsement estimates the post-meal timing of the exercise, which coincides with the post-meal blood glucose peak approaching its maximum, FIG. 37 and FIG. 38.

The calculation for the start time of the post-meal exercises, as well as the proposed energy expenditure of the exercise is derived from the glycemic indices (GI) of the food items of the meal and the relations between the carbs and calories of food items at different levels of glycemic indices. In practice, GI of food items is classified as: low, medium and high. The borders between these three classes are given in the literature by: GI low: giLowε[0, 54]; GI medium: giMidε[55, 69]; and GI high: giMH≧70. For high GI, the start time is recommended as 30 minutes after the meal.

The BG peak reduction procedure first identifies the amounts of calories and carbs of food items with GI low, medium and high. They are marked as calLow (calories of foods with low GI), calMid (medium calories), calHi (high calories), carbLow (low carbs), carbMid (medium carbs) and carbHi (high carbs), by going over the GIs of every food item in a meal. Then, the maximal GIs of food items for items of high and medium GIs are found and recorded as giMax and giMed, respectively. These valued are used to obtain carb ratios carbRatioHi and carbRatioMid between the high and low GI carbs and between the medium an low carbs, respectively. The amounts of calories to expend, calsToBum is given as:

```
if ( giMax ≧ giHM ) {
  carbRatioHi = ( carbHi − carbLow ) / carbTotal;
  cal4H = calTotal − calMid;
  if ( carbRatioHi > 0.2 ) {
  calsToBurn =
  = 0.25 × (carbHi − carbLow) × (glHi / glTotal) × (calTotal / carbTotal);
  if ( calsToBurn > cal4H / 2)
    calsToBurn = 0.5 × (carbTotal − carbLow) × (calTotal / carbTotal);
  }
}
```

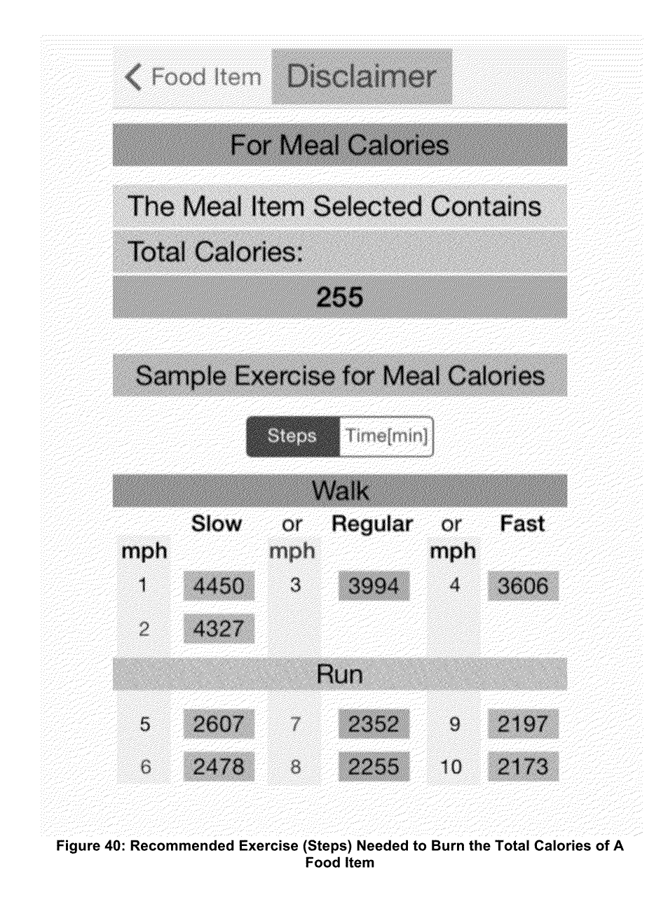

Note that when the carbs ratio is low, then the post-meal blood glucose would not be significantly elevated, and no recommendations for the post-meal exercises will be issued, FIG. 39. Therefore, that case was not considered in the method for addressing the post-meal blood glucose level.

The Carb Impact shows that the healthy way of keeping the daily caloric balance in the targeted area is by correctly timing the food consumption and the exercise routines. For example, if the user exercises a lot in the morning, and restricts the calories consumption during the day, the consumption of the food with high carb count, glycemic load and glycemic index would still cause an elevated post-meal blood glucose level, and possible weight gain and other diabetes related consequences, even if the daily caloric balance is sustained in its target region.

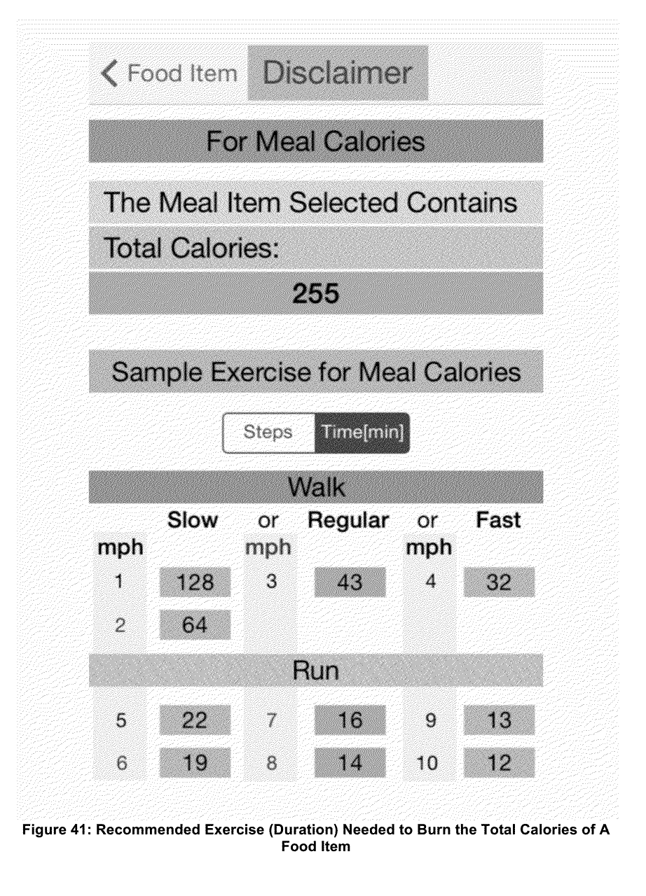

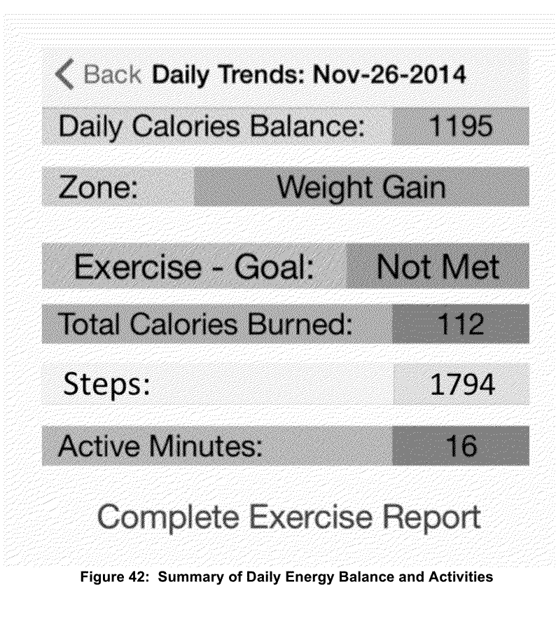

Finally, for each food item, the user is given the Calory Impact, which is the amount of exercise (steps) and duration of physical activities needed to burn the complete caloric content of the food item, FIG. 40 and FIG. 41.

Given the progress in food consumption and the exercise level, it is possible to realistically monitor the weight-loss progress. Tracking weight loss progress is achieved by calculating the required energy deficit, obtained from the weight loss goal pro-rated for the time of the day. Energy balance between the energy ingested by food and the expended energy is visually compared in a single graph against the weight loss goal, FIG. 21 and FIG. 22. Furthermore, energy consumed is presented by a progress bar with an arrow advancing towards the right, while the food intake is presented as a progress bar with an arrow advancing towards the left from the point of the current energy expenditure amount. This combination allows easy verification whether the current weight loss goal fulfillment is positive or negative, and hence whether the current status leads to weight loss or a gain, FIG. 21 and FIG. 22. In addition to the sign of the weight loss progress, the absolute value is compared to the predetermined weight loss gain for a given time of the day.

Daily Trends.

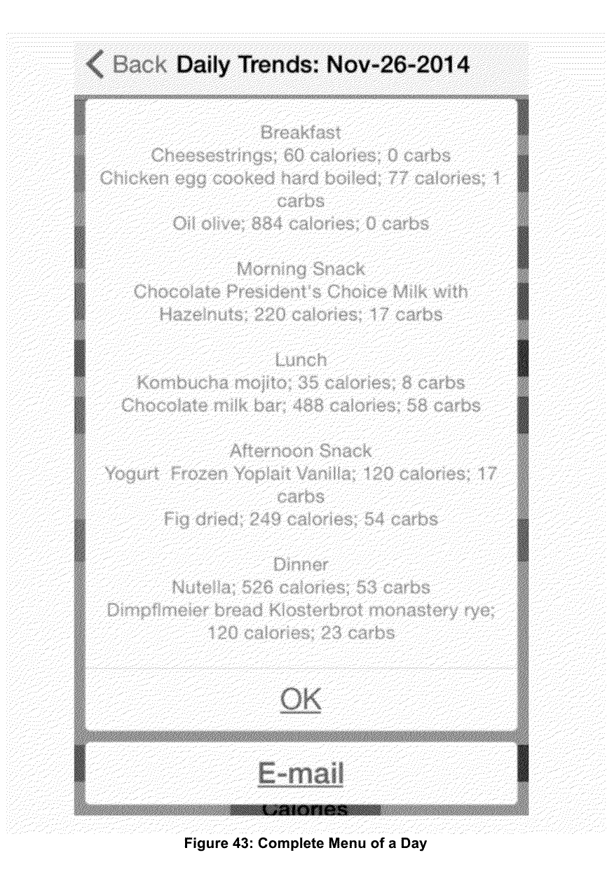

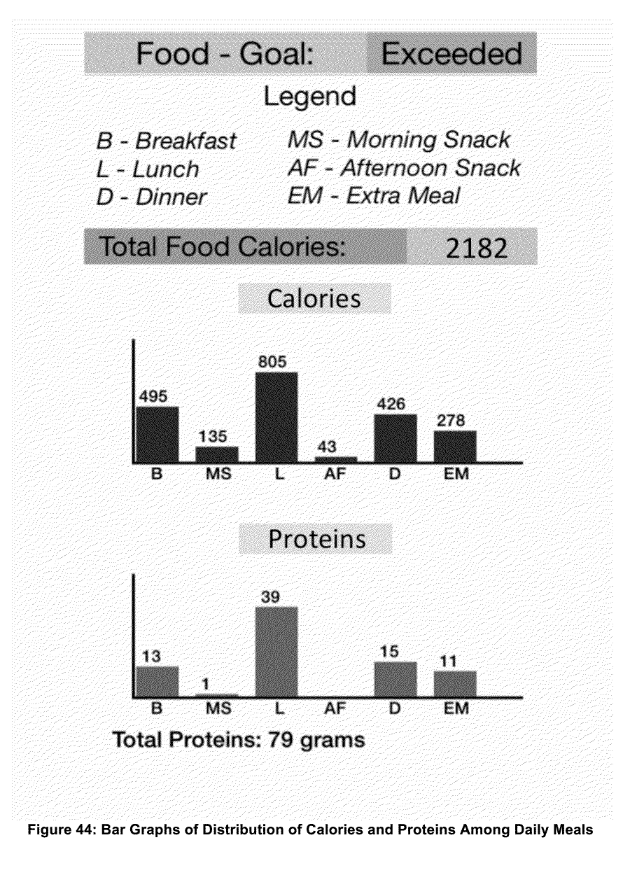

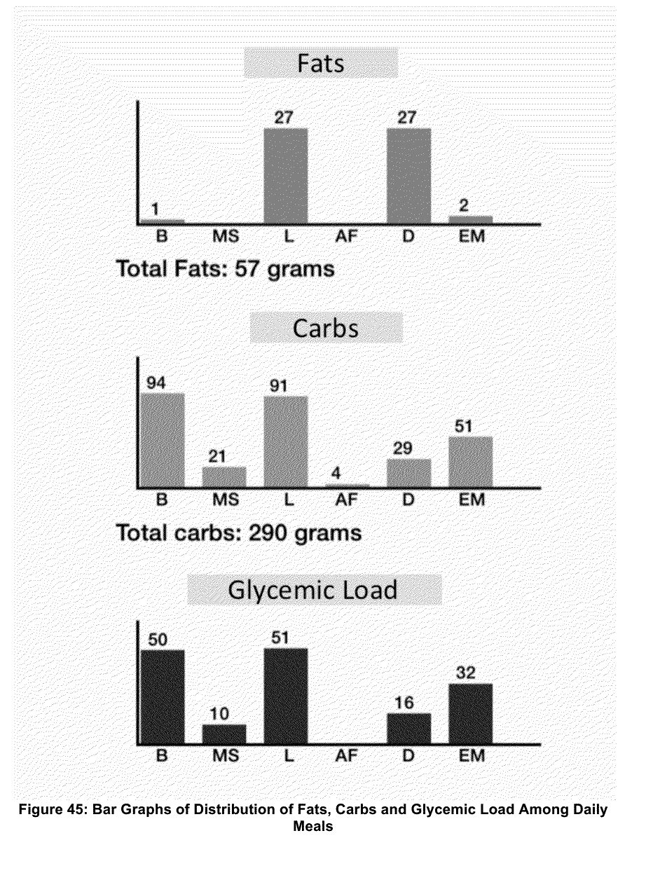

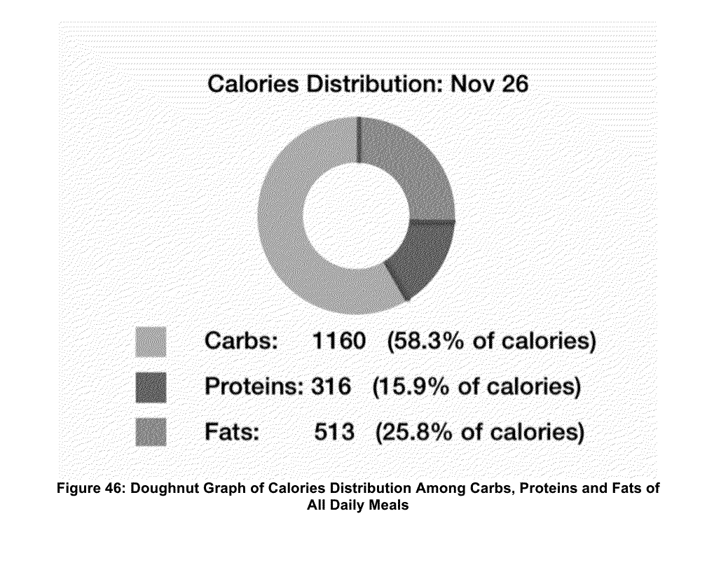

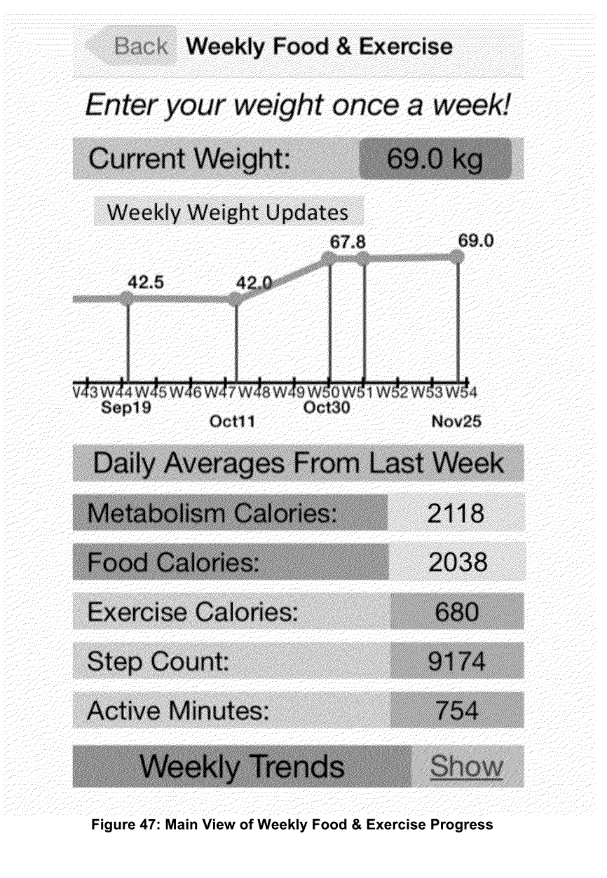

Daily Trends illustrates daily progress in energy budget and nutrient consumes. Information in conveyed on a separate visual display. In the compact form, the tool reports:
Daily calories balance (food calories minus exercise and metabolism calories), FIG. 42;
Weigh loss/gain zone: weight-loss if calories balance is negative, weigh-gain if the calories balance is positive, FIG. 42;
Summary of total daily exercises: calories burned, steps, active minutes, FIG. 42,
Link to the pedometer page with detailed analysis of daily activities, [0085]-[0092],
Link to the popup complete daily menu. For each consumed meal the user obtains the registry of all the food items consumed with the listing of calories and carbs content of each food item, FIG. 43. The menu can be automatically sent to the selected recipients.
Bar graphs summarizing food nutrients such as calories, proteins, fats, carbs and glycemic load displayed for each meal (breakfast, morning snack, lunch, afternoon snack, dinner, extra meals), FIG. 44 and FIG. 45;
Doughnut-diagram display of daily food calories distribution among carbs, proteins and fats, FIG. 46.

By relating daily physical activities with daily meals nutrients, Daily Trends view allows the user to analyze his/her habitual behavior and provide insight into possible improvements. All exercise related data is ported from dataset updated automatically by the pedometer. All food data is taken from dataset based on the daily meal updates by the user.

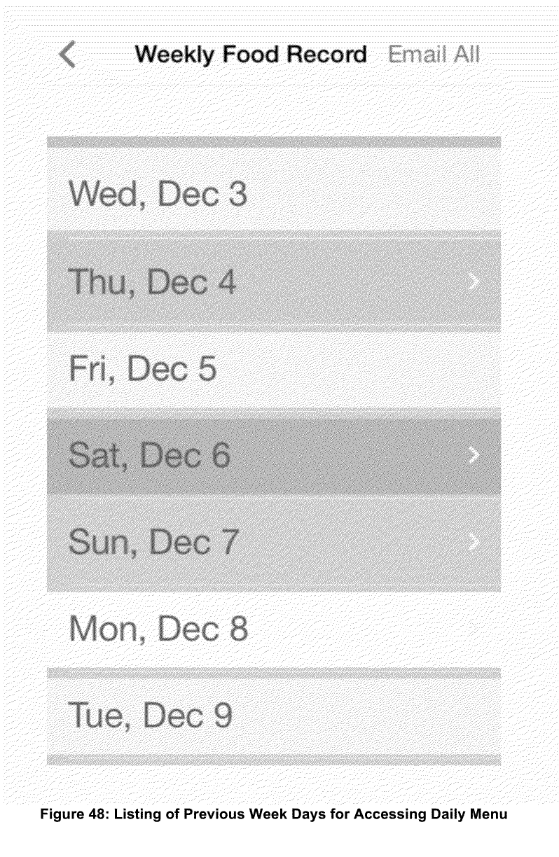

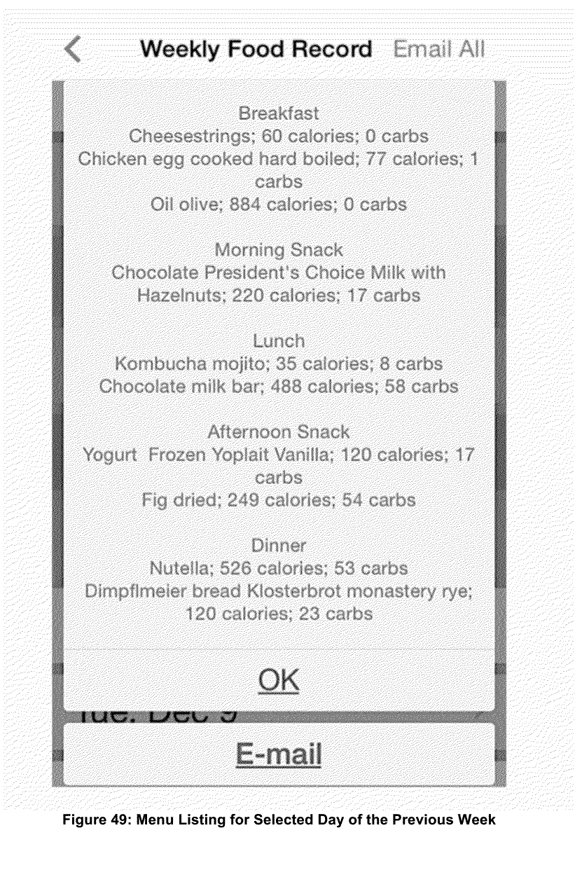

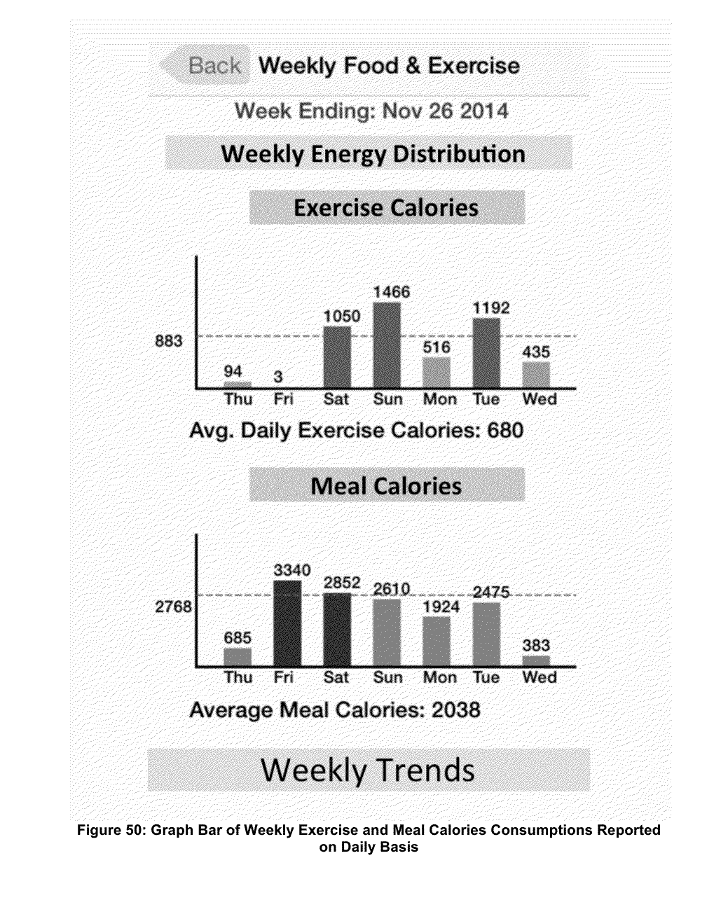

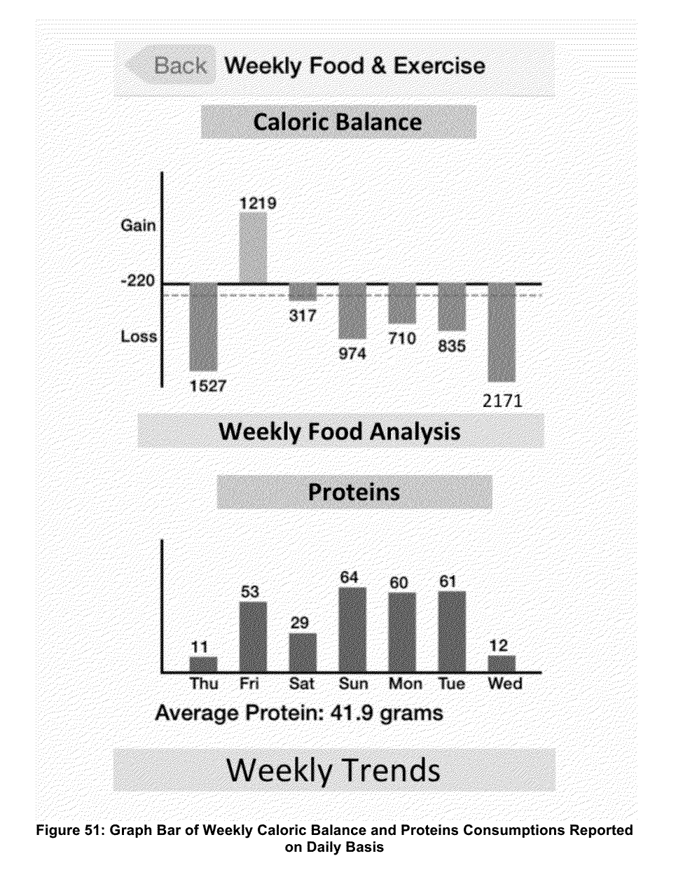

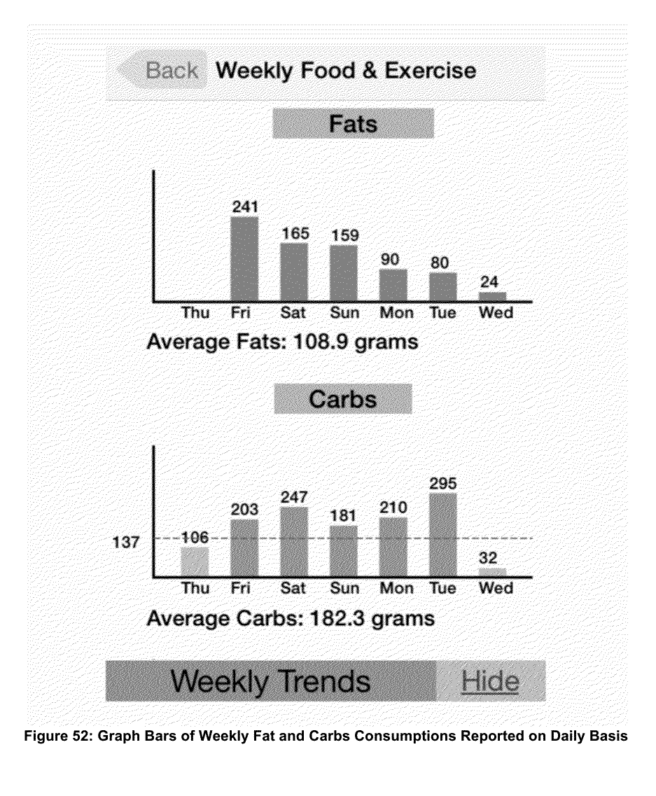

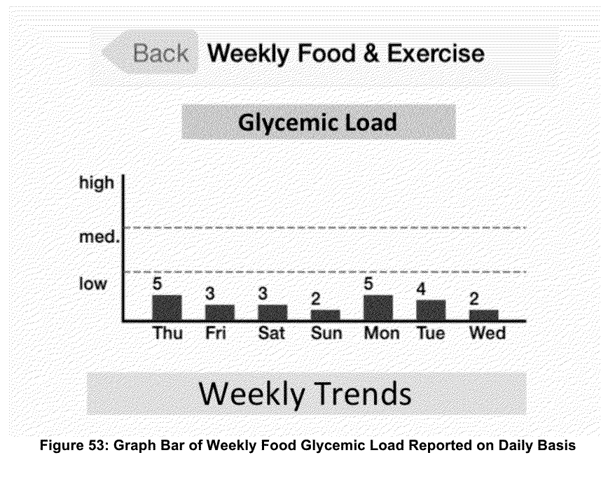

Weekly Trends facilitates better analysis of impact of exercise and food on wellness. It informs the users about the daily balances and food intake details in the last seven days, as well as the visual display of the weight tracking data. The analysis display on that view comprises of:
Weight Analysis. The user enters his/her weekly weight. Weekly weights are displayed as a graph, FIG. 47. The weight data can be updated at most once a week in that view, either by manually entering the data or by collecting the output of a connected scale, such as the Bluetooth Low Energy (BLE) scale. The weight graph indicates recorded weights together with the time each weight was entered to the system. Weekly weight updates can be traced on the graph up to the first entry, by scrolling the display.
   Weekly weight updates, particularly when the weight changes are anticipated are crucial for the correct functioning of the whole system, and not only the weight-loss program. The reason is that the current user's weight is used among others to determine the daily metabolic calories. Further, current weight is used in determining the exercise energy expenditure. Therefore, any updates to the current weight should be recorded to allow the precise estimation of daily metabolism, exercise, and in consequently, calories balance. Note that it is enough to enter the weight change in this entry. The change will be automatically transmitted to Personal Settings, where all system-required user's personal parameters are displayed.
   Similarly, if the user (a child) experience high changes due to growth process, then these changes should also be entered into the system. As growth changes happen generally not on the regular weekly basis, then the only place to enter high changes is the Personal Setups.
Food Menu from Last Week. The user can view the complete food menu from last week, listed day by day, FIG. 48. By selecting a day of interest from the listing in FIG. 48, the user can view the complete menu for that day. The menu includes the names of food items, serving sizes, as well as calories and carbs contents, FIG. 49.
Daily Averages from Last Week. Calculated based on the daily data traced by the system, the user is presented with daily averages of:
   Metabolism calories. Metabolism calories depend among others on the current user's weight, FIG. 47. Any weekly weight changes affect metabolism calories burned daily. As these calories are an important factor in the overall daily energy balance, it is important that the user knows the value of metabolism calories as close as possible to the actual one, such that better meal and exercise routines can be planned.
   Food calories. This entry presents the average amount of calories consumed daily in the last week, calculated based on the daily food consumption registered in the passing week, FIG. 47.
   Exercise calories. Daily average exercise calories are calculated based on the total last week exercise calories determined by the pedometer, FIG. 47.
   Step count. Daily average step count is determined based on the total step count in the passing week established by the pedometer, FIG. 47.
   Active minutes. Daily average active minutes are established based on the total active minutes registered by the pedometer in the passing week, FIG. 47.
Weekly Trends Analysis. If the user wants to obtain more detailed weekly report, by pressing a “Weekly Trends” button, a scrollable view is presented with the daily details for the last seven days. This detailed weekly view contains bar diagrams for daily exercise calories, for daily meal calories, as well as the energy balance between the two. Further, for the weekly food details, additional graph bars are displayed for the daily protein, fat, carbohydrate and glycemic load data. Weekly Trends Analysis entry prompts the user to the set of bar graphs illustrating:
   Exercise calories. The bar graph shows daily exercise calories of the passing week, FIG. 50. Value in calories is presented over each bar. Bars representing daily exercises that fall below the daily exercise goal are marked in lighter shade than the bars for exercise exceeding the goal. Daily average is written below the graph.
   Meal calories. The bar graph shows daily food calories consumed in the passing week. The numerical value of the calories consumed in all meals in a given day is presented over each bar. Bars representing daily calories consumptions that fall below the daily goal are marked in lighter shade than the bars for food calories on target or exceeding the daily goal. To summarize the average daily food calories consumption, the daily average is written below the graph, FIG. 50.
   Caloric balance. The bar graph indicates calories balance values, calculated by subtracting daily exercise and metabolism calories from daily meal calories. Bars above the horizontal Daily Coordinates Line indicate positive caloric balance (a possible weight-gain scenario), while bars below the horizontal Daily Coordinates Line point to the negative caloric balance, and as a result the possible weight-loss. Bars representing daily caloric balance that fall below the daily goal are marked in lighter shade than the bars for balance on target or exceeding the daily goal. The summary of the average daily carbs consumption is displayed below the carbs bar graph, FIG. 51.
   Proteins. The graph bar records the amount of proteins accumulated in all daily meals. The summary of the average daily protein consumption is displayed below the protein bar graph, FIG. 51.
   Fats. The graph bar presents the amount of fats accumulated in all daily meals. The summary of the average daily fat consumption is displayed below the fats bar graph, FIG. 52.
   Carbs. The graph bar features the amount of carbs consumed each day in all meals over the passing week. Daily Carbs goal is presented in the graph. Bars representing daily carbs that fall below the daily goal are marked in lighter shade than the bars for carbs intake on target or exceeding the daily goal. The summary of the average daily carbs consumption is displayed below the carbs bar graph, FIG. 52.
   Glycemic load. The graph bar implying the approximate accumulative glycemic load of daily-consumed food, FIG. 53.

Weekly Weight-Loss.

This entry is dedicated to the users having the weight-loss program incorporated into their daily routines. It is designed to promote better understanding and feasible assumptions for a weight-loss plan. The operation of this page is based on the weight-loss goal set by the user, data provided by the pedometer and input from meal pages regarding daily food intake and its nutrients profile.

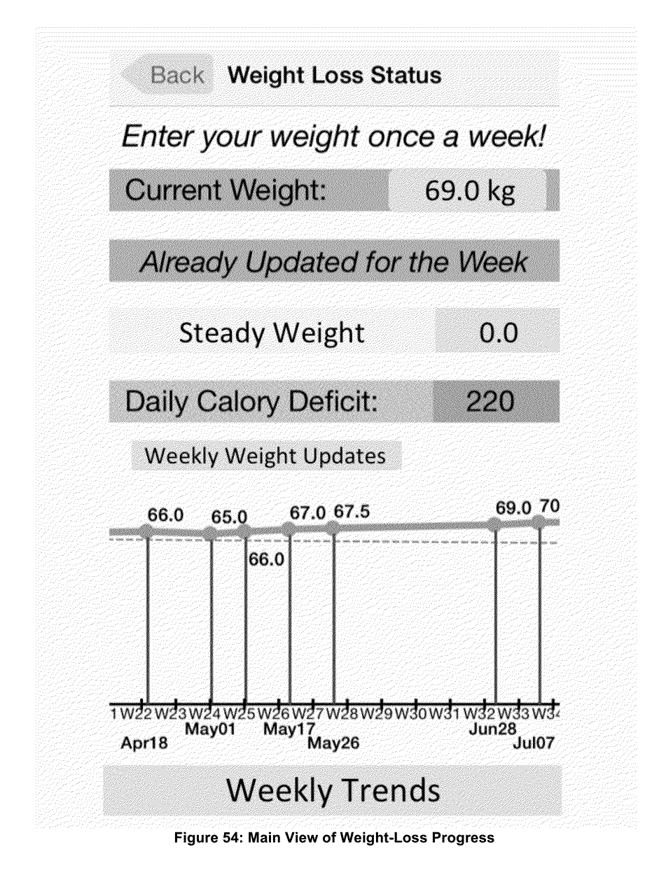

The main Weigh-Loss entry is presented in FIG. 54. In the page, the user can see his/her current weight (numerical display).
Weight-loss statistics. The user is asked to enter his/her weight once a week, FIG. 54. Based on the current weight, the system determines the weekly weight loss as well as daily calories deficit for next week.
   Weekly weight updates, particularly when the weight changes are anticipated, are crucial for the correct functioning of the weight loss regime, and the proposed weight-loss method. The reason is that the current user's weight is used among others to determine the daily metabolic calories. Further, current weight is used in determining the exercise energy expenditure. Therefore, any updates to the current weight should be recorded to allow the system the precise estimation of daily metabolism, exercise, and in consequence, caloric balance. Note that it is enough to enter the weight change in this entry. The change will be automatically transmitted to Personal Settings, where all system-required user's personal parameters are displayed.
Weight-loss Progress. The tool informs the user about the weight-loss progress. Here the three options are possible: Weight Gain (with numerical listing of extra weight gained in the last week), Steady Weight (the weight gain/loss is 0 in the last week), and Weight Loss (numerical display of the weight lost in the last week), FIG. 54.
Daily Calories Deficit. Daily Calories Deficit informs the user about the daily calories balance, which would promote the weight loss. The initial calories deficit is established in the Weight-Loss Goal entry described in the next section. The deficit is calculated based in the current user's weight, duration of the weight-loss program and desired weight-loss expressed in kilograms or pounds. In the duration of the weight-loss program, the users weight would change. Further, in some weeks the user may experience larger or smaller weekly weight loss than initially planned. Above factors are considered, leading to possible adjustments in daily calories deficit for the coming week, FIG. 54.
   Given the daily calories deficit, planned daily exercise calories and metabolism calories, the user is presented with the amount of food calories, which he/she can consume daily and still promote the weight-loss goal. Upon changing the daily calories deficit, and in consequence the total daily calories consumption goal, the user is informed to adjust each daily meal calories goal to match the new food caloric balance.
Weekly Weight Updates Graph. Once entered, the new weight is displayed on the sliding graph recording all the weighs from the beginning of the weight-loss program, FIG. 54. Above each weight entry in the graph, its numerical value is displayed. The weight progression in the graph is presented for active weeks of the weight-loss program.
Weekly Trends Analysis. This entry is identical to that provided in Weekly Trends [00121], and presents information related to the previous week's daily exercise calories, meal calories, caloric balance, proteins, fats, carbs and glycemic load. All of the above data is presented using bar graphs, FIG. 51, FIG. 52 and FIG. 53. This entrance is presented in the Weekly Weight-Loss section for completeness of weight-loss data analysis.

Weight-Loss Goal.

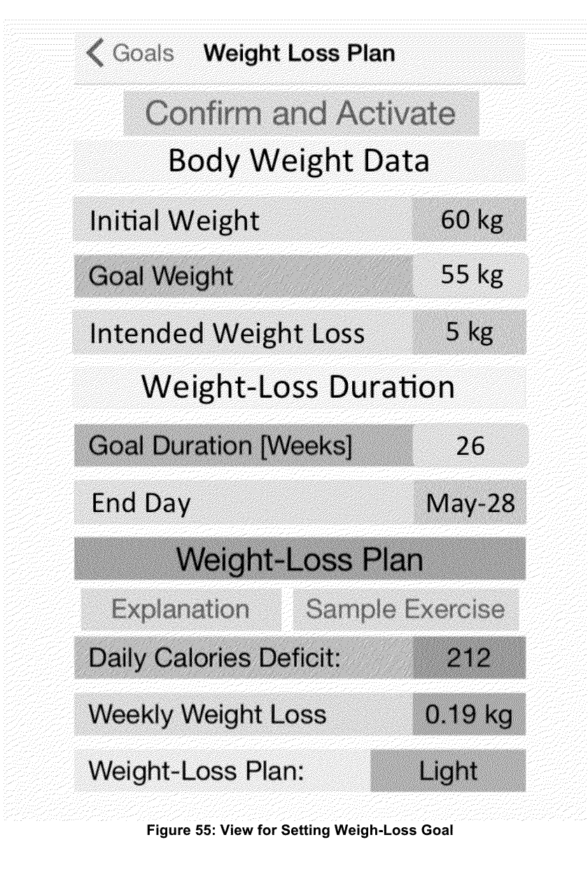

The entry is designed to refine a weight-loss plan. This entry determines: the end day of the weight-loss plan, the daily caloric deficit needed to reach the desired weight and weekly weight-loss reported in the measurement units selected by the user, FIG. 55. Accordingly, the entry is separated into three sections:
Body Weight Data. The tool lists the initial weight of the user taken from the Personal Settings [00126]. The user has to enter the goal weight. The tool displays then the intended amount of weight to be lost. All weight measurements are presented in the user-selected units (standard or imperial), FIG. 55.
Weigh-Loss Duration. The user must specify the duration in weeks of the weight-loss plan. Since the weight-loss plan starts immediately, the tool determines the date of the completion of the plan, FIG. 55.
Weight-Loss Plan. The tool reports Daily Calories Deficit, Weekly Weight Loss and the severity of the weight-loss plan, FIG. 55.
   Daily Calories Deficit represents the daily negative caloric balance needed to obtain the assumed weekly weight loss. As one of the factors in determining the daily calories deficit is the current weight of the user, when the weight changes, daily calories deficit for a given week would also change. Sample exercises are indicated for which the energy expenditure is equivalent to the daily calories deficit.
   Weekly Weight Loss is calculated based on the current weight, goal weight and the remaining amount of planned weeks to reach that goal.
   Weight-Loss Plan determines the severity of the plan based on the intended weekly weight loss. The options are: No Plan (0 calories deficit per day), Light (˜250 calories deficit per day), Medium (˜500 calories deficit per day), Aggressive (˜750 calories deficit per day), and Too Hard (over 750 calorie deficit per day)
   Sample Exercises is the link to the Exercise Calories Goal, where examples are provided for steps and duration that burn the daily calories deficit specified by the weight-loss plan.

Settings.

The tool facilitates four types of settings: Personal Settings, Measurement Units, E-mail Recipients and Carbs Counting.

Personal Settings.

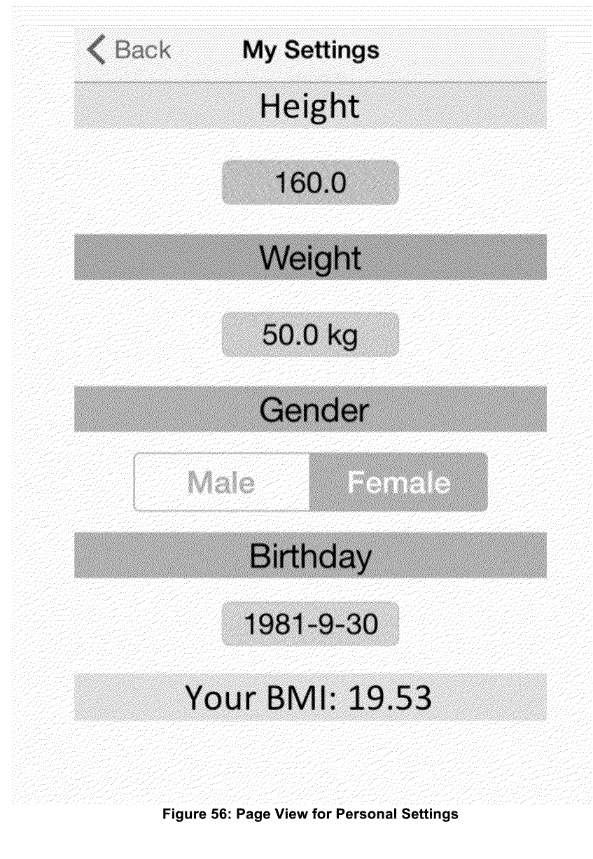

In this entry the user specifies his/her height, weight, gender and birthday, FIG. 56. This entry is important for the correctness of the operation of the pedometer, as the above parameters are used in the estimation of the stride length, BMR (basal metabolism rate), as well as energy expenditure of all exercises registered by the pedometer.

Measurement Units.

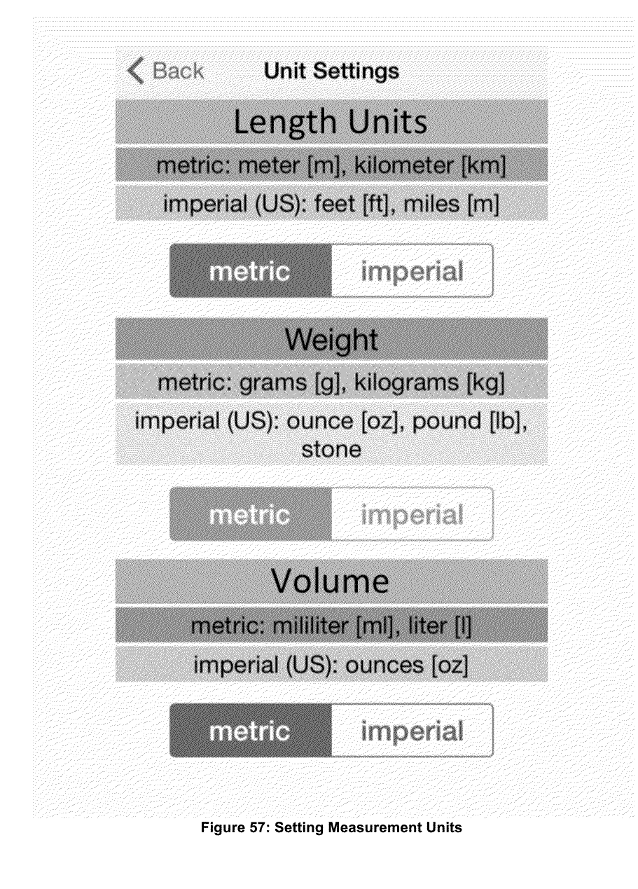

The three basic measurement units: length, weight and volume can be expressed in metric and in imperial system. Mixing of units (for example length in metric and weight in imperial units) is allowed, FIG. 57.

E-Mail Recipients.

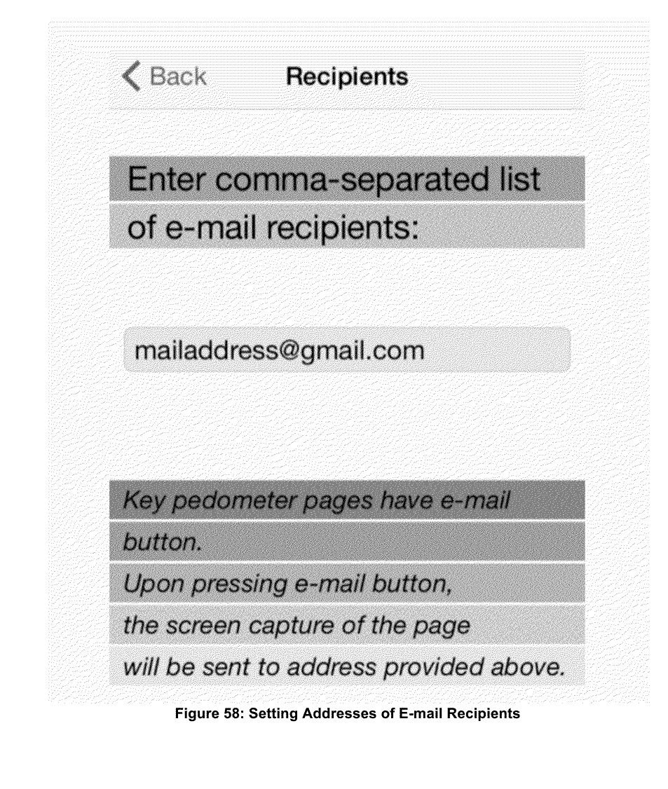

The tool supports sending some data via e-mail to selected recipients, FIG. 58. This includes entries such as: summary of up to date daily physical activities, physical activates in selected time interval, nutrients analysis of a complete meal, daily trends, weekly trends and weekly weight-loss. The screen capture of the above pages can be sent automatically to the selected recipients. The e-mail addresses of the recipients must be specified in the E-mail Recipients Setting.

Carbs Counting.

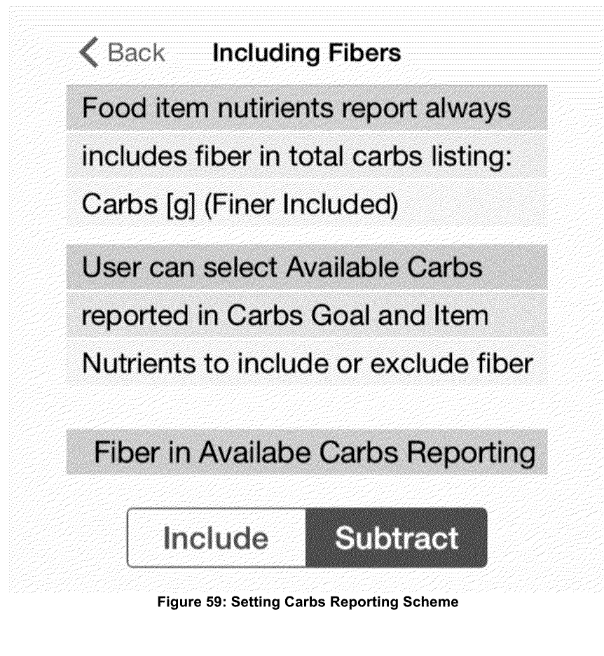

The user can select fiber to be included or excluded in the total reporting of available carbs for a give meal, as well as the total available carbs consumed in the meal, FIG. 59. Note that in addition to the available carbs, which based on the user's preferences would or would not include fiber, the tool will report explicitly, for a given food item or the whole meal, the total carbs I that include fiber.

### REFERENCES

[1] G. Trapp, B. Giles-Corti, M. Bulsara, H. Christian, A. Timperio, G. McCormack and K. Villaneuva, “Measurement of Children's Physical Activity using a Pedometer with a Built-in Memory”, Journal of Science and Medicine in Sport, Vol. 16, No. 3, May 2013, pp. 222-226.
[2] C. Tudor-Locke, C. L. Craig, W. J. Brown, S. A. Clemens, K. de Cocker, B. Giles-Corti, Y. Hatano, S. Inoue, S. M. Matsudo, N. Mutrie, J. M. Oppert, D. A. Rowe, M. D. Schmidt, G. M. Schofled, J. C. Spencer, P. J. Teixeira, M. A. Tully, S. N. Blair, “How Many Steps/Day are Enough? For Adults”, Int Journal Behav. Nutr Phys Act., 8:79. Doi: 10.1186/1479-5868-8-79, Jul. 28, 2011.
[3] K. Radecka and Z. Zilic, “Accurate Step Counting Pedometer for Children, Adults and Elderly”, U.S. patent application Ser. No. 14/324,055, Filed Jul. 3, 2014.
[4] A. M. Roza and H. M. Shizgal, “The Harris-Benedict Equation Reevaluated: the Resting Energy Requirements and the Body Cell Mass”, American Journal of Clinical Nutrition, vol. 40, pp. 168-184, 1984.
[5] P. H. Sessoms, “Step by Step: A Study of Step Length in Able-bodied Persons, Race Walkers, and Persons with Amputation”, Ph.D. Dissertation, Northwestern University, 2008.
[6] D. Landers, “Digital Weight Loss Aid”, US Patent Application, US2012/0221495, Filed August 2012.
[7] S. Abraham and C. Ferguson, “Method and Composition for Improved Muscle Performance”, U.S. Pat. No. 8,703,719, April 2014.
[8] E. H. Mathews, “Apparatus and Method for Predicting the Effect of Ingested Foodstuff or Exercise on Blood Sugar Level of Patient and Suggesting a Corrective Action”, PCT Patent Application, PCT/ZA2006/000010, January 2006.
[9] J. Mault and J. Sanderson, “Closed Loop Glycemic Index System”, US patent application U.S. Ser. No. 10/333,383, 2003.
[10] F. Kaufman, “Methods for Suppressing Appetite and Enhancing Exercise and Recovery”, PCT Patent, PCT/US2000/021082, 2012.
[11] N. Chakrabarty, “Method for Managing Obesity, Diabetes and other Glucose-spike-induced Diseases”, U.S. Pat. No. 8,589,082, 2013.

1. A method for wellness and health improvement through control of lifestyle and weight management during daily activities and exercise for healthy and persons susceptible to diabetes; the method: a) applies the energy expenditure estimation; b) food entry and energy consumption; c) monitors the daily caloric balance, and comprises:
I. A step counting method based on the data in the frame selected by the energy detection that recognizes: walking steps in speeds: slow (1 and 2 mph), regular (3 mph) and fast (4 mph); alternatively, the walk speeds in metric system are: slow (2.0 and 3.5 km/h), regular (5.0 km/h) and fast (6.5 km/h); run steps in speeds: slow (5 and 6 mph), regular (7 and 8 mph) and fast (9 and 10 or greater mph); alternatively, the corresponding types of run in metric system are: slow (8.0 and 9.5 km/h), regular (11.0 and 12.5 km/h) and fast (14.0 and 16.0 or greater km/h)
II. A method for obtaining the energy expenditure for daily exercise;
III. A method for setting a short-term (single execution) exercise goals in terms of steps including type (walking or running) and speed of execution, duration including type (walking or running) and speed of execution, or total calories to be exhorted during the exercise; the execution of the short-term exercise goal is monitored and the user is informed when the goal is reached;
IV. A method for inputting food entries for daily meals, and staying within the goal brackets for calories and carbs consumption per meal, as well as daily caloric expenditure;
V. A method for specifying the daily goals for steps, exercise calories and exercise duration;
VI. A method for specifying meal goals in terms of calories and carbs;
VII. A method of reporting nutrients of each food item and the total meal; whereas the carb content of a food item, as well as the total meal, is presented to either include or exclude fiber (depending on the preferences set by the user); further, included is the report of the total carb content (fiber included) of the food item and a complete meal;
VIII. A method allowing selection of the consumed item quantity based on: weight, calories, carbs or portion size;
IX. A method of reporting the nutrients of the complete meal numerically and graphically by using a doughnut graph for the percentage of fats, carbs and proteins in the total calories of the meal;
X. A method of aligning exercise calories, food calories and daily caloric balance on one display allowing the user to track the total caloric balance through the day.

2. A method in claim 1, whereas the tracking of exercise levels displays visually the food intake and weight loss prospectus, all relative to their respective goals, and indicative of whether the current progress leads to weight loss or weight gain.

3. A method in claim 1, whereas the reporting of the exercise progress is done by means of progress bars that do not saturate at 100%, but relatively extend pass that, to visually and quantitatively express the fulfillment of exercise relative to goals such as calories, steps, distance and duration goals.

4. A method in claim 1 for weight-loss management, where the daily caloric deficit is established to obtain the goal weight in the goal time; the daily caloric deficit is determined based on the personal metabolism, planned daily exercise level as well as planned weekly weight loss; as metabolism is affected by the current weight, the daily caloric deficit is updated once a week based on the new current user's weight.

5. A method for the updates of meal calories goals in an exercise and meal tracking device comprising:
I. Initial meal calories goals entry;
II. An information entry on the calories consumed so far;
III. A current energy expenditure of the day;
IV. A method for declaring the bonus calories for a current meal goal that accounts for caloric imbalance of previous meals and exercise exceeding the daily goal; the calories consumption deficit with respect to previous meals goals is given as a positive bonus for the goal of the currently considered meal; the excess of the caloric consumption in the previous meals exceeded the meals goals is presented as a negative bonus for the goal of the current meal;
V. A method by which, if the exercise calories exceeded the daily goal at the time of the current meal creation, then the exercise calories excess is given as a positive bonus for the goal of the present meal; conversely, the fact that the exercise calories goal is not reached at the time of the current meal creation has no influence on the adjusting of the current meal goal.

6. A method advising the exercise level and timing following food intake to minimize post-meal blood glucose peaks based on the analysis of the profile of the consumed food comprising:
I. A meal entry information means including:
a. Meal glycemic index information entry;
b. Meal glycemic load information entry;
c. Calories and carbs information;
II. A method that incorporates the impact of the meal glycemic index on the time of the post-meal blood glucose spike to issue an advice for the user is regarding the beginning of the exercise as well as its type and duration (walk, run, step count and speed) to help reducing the post-meal blood glucose peak.
